(19) United States
(12) Patent Application Publication
(10) Pub. No.: US 2001/0031744 A1
(43) Pub. Date: Oct. 18, 2001

(54) Compositions and methods for prevention and treatment of chronic diseases and disorders including the complications of diabetes mellitus

(76) Inventor: John V. Kosbab, Dillon, CO (US)

Correspondence Address: GREENLEE WINNER and SULLIVAN, P.C., Suite 201, 5370 Manhattan Circle, Boulder, CO, 80303, US

(21) Appl. No.: 09/827,251

(22) Filed: Apr. 5, 2001

Related U.S. Application Data
(63) Continuation of application No. 09/018,273, filed on Feb. 4, 1998, now abandoned.
(60) Provisional application No. 60/037,084, filed on Feb. 4, 1997.
(60) Provisional application No. 60/043,262, filed on Apr. 17, 1997.

(51) Int. Cl. A61K 35/78; A61K 31/737

(52) U.S. Cl. 514/54; 514/62; 424/729; 424/770; 424/732; 514/458; 514/725; 514/474

(57) ABSTRACT

This invention relates to nutrient and therapeutic compositions for treatment and prevention of symptoms and disease conditions associated with microangiopathy and macroangiopathy and to methods using the compositions. In particular, the invention relates to compositions useful in the treatment of diabetic retinopathy and nephropathy, to compositions useful in the treatment of other retinal disorders including macular degeneration and cataracts, to compositions useful in wound healing, to compositions useful for treatment and prevention of neuropathy, to compositions useful for treatment and prevention of cardiovascular disease and to compositions useful for the treatment and prevention of dental and periodontal disorders.

### CROSS-REFERENCE TO RELATED APPLICATIONS

This application is a continuation of U.S. patent application Ser. No. 09/018,273, filed Feb. 4, 1998 which claims priority to U.S. Provisional application Ser. No. 60/037,084, filed Feb. 4, 1997 and Ser. No. 60/043,262, filed Apr. 17, 1997, all of which are incorporated by reference in their entirety herein.

### FIELD OF THE INVENTION

This invention relates to the use of nutrient and therapeutic compositions to ameliorate the disease symptoms and conditions associated with vascular and capillary disorders: microangiopathy and macroangiopathy. Compositions of this invention include antioxidants, neovascular regulators, promoters or cofactors involved in collagen synthesis, as well as vitamins and minerals to supplement deficiencies.

### BACKGROUND OF THE INVENTION

Vascular degeneration, both macroangiopathy and microangiopathy (capillary degeneration), is believed to be the root cause of a variety of degenerative disease conditions that effect a substantial portion of the population. Vascular degeneration is directly associated with cardiovascular disease, atherosclerosis and plaque deposition and indirectly associated with degenerative conditions of the retina (including retinopathy), kidney (nephropathy) and nervous system (neuropathy), as well as skin ulcers.

A broad variety of treatments have been proposed for conditions associated with microangiopathy, particularly for retinopathy, nephropathy, neuropathy and skin ulcers. Similarly, a variety of treatments and preventive formulas have been proposed for cardiovascular disease. These treatments have met with limited or no success. In some cases, allergic reactions, side effects, drug interactions, or the impracticality of drug therapy have posed serious problems.

There is clearly a serious and substantial need for methods of treatment which slows or reverses, even temporarily, the onset of symptoms as described above which affect such large numbers of people. There is also clearly a need for methods for preventing the onset or worsening of such conditions.

The present invention is directed to nutrient and therapeutic compositions for the treatment and prevention of disease conditions associated with vascular and capillary degeneration. The compositions provided herein are useful in treating a variety of conditions including: cardiovascular disease, disease of the retina, nephropathy, and neuropathy. Compositions of this invention are also useful in wound treatment and in the treatment and prevention of dental and periodontal disease. Retinopathy, nephropathy, neuropathy, recurrent, slow-to-heal wounds, and gum disease and tooth loss are complications of diabetes. Formulas of this invention include those that are specifically formulated to improve diabetic complications.

Compositions of this invention include antioxidants, neovascular regulators, factors that promote or stimulate collagen synthesis and provide nutrients, vitamins and other components to provide nutritional balance. Additional components provide benefit to diabetics. These compositions are directed to the improvement of symptoms and disease conditions by correcting vascular degeneration and by maintaining healthy vascular and capillary tissue.

The multi-component compositions and methods of treatment of this invention differ from previous proposed treatments in that they are intended to simultaneously ameliorate multiple related factors that are believed to contribute to the disease conditions. Most previous therapeutic compositions for treatment of diabetic complications, including retinopathy and nephropathy, have attempted to treat only one aspect of the disease state.

### SUMMARY OF THE INVENTION

This invention relates to the use of nutrient and therapeutic compositions to ameliorate disease conditions, symptoms and disorders resulting, at least in part, from tissue and cell damage due to oxidative stress and the breakdown of collagen in tissues. In particular, the nutrient and therapeutic compositions of this invention are useful in the prevention and treatment of symptoms and disease conditions associated with vascular and capillary impairment, including macroangiopathy and microangiopathy. The invention specifically provides compositions and methods for the prevention and treatment of diabetic complications, retinopathy, nephropathy, neuropathy, cardiovascular disorders and diseases, slow-to heal or recurrent wounds and gum and tooth disorders including periodontal disease. All of these disorders are believed to share common etiological factors, so that compositions containing related ingredients are effective for treatment and/or prevention of these disorders and conditions.

In a specific embodiment the present invention provides therapeutic and nutrient compositions and treatment methods using those compositions for ameliorating conditions and symptoms associated with microangiopathy, particularly the complications of diabetes mellitus associated with microangiopathy. More particularly, the methods and compositions of this invention are useful in the amelioration and treatment of diabetic retinopathy and nephropathy. The methods and compositions of this invention are further useful in the treatment of other degenerative ocular conditions such as macular degeneration, cataracts and glaucoma.

In another specific embodiment, the present invention provides therapeutic and nutrient compositions and treatment methods using those compositions for wound healing, also providing for treatment of recurring and/or slow-to-heal wounds, including among others the treatment of decubitus ulcers. Compositions of this invention can be administered by a variety of routes to an individual having slow-to-heal or recurrent wounds, preferred compositions are for oral administration. The invention also provides wound healing formulations for topical application to wound sites, particularly in the form of ointments. Nutrient compositions useful for prevention of wound development, or for preventing recurrence of slow-to heal wounds in an individual at risk for development of such wounds are also provided. The formulas provided herein for wound healing include those that are adapted for use by diabetics to provide additional benefits for the treatment or prevention of diabetic complications.

In a third specific embodiment, the present invention provides therapeutic and nutrient compositions and treatment methods using those compositions for the symptoms and disease conditions associated with neuropathy. Compositions of this invention can be administered by a variety of routes to an individual having neuropathy, preferred compositions are for oral administration. The invention also provides formulations for topical application for relief of the symptoms of neuropathy, including pain relief. Nutrient compositions useful for prevention of neuropathy or for preventing recurrence of symptoms of neuropathy in an individual at risk for development of such symptoms is also provided. The formulas provided herein for neuropathy include those that are adapted for use by diabetics to provide additional benefits for the treatment or prevention of diabetic complications.

In a fourth specific embodiment, therapeutic and nutrient compositions and treatment methods using those compositions are provided for conditions associated with macroangiopathy (vascular degeneration), particularly for the treatment of cardiovascular disease. Compositions of this invention can be administered by a variety of routes to an individual having symptoms and conditions associated with macroangiopathy, preferred compositions are for oral administration. Nutrient compositions for the prevention of cardiovascular disease are provided. The formulas provided herein for cardiovascular disease include those that are adapted for use by diabetics to provide additional benefits for the treatment or prevention of diabetic complications.

In yet another specific embodiment, therapeutic and nutrient compositions and treatment methods using those compositions are provided for dental and periodontal disease. Compositions of this invention can be administered by a variety of routes to an individual having symptoms and conditions associated with tooth and gum disease, preferred compositions are for oral administration. Nutrient compositions for the prevention of tooth and gum disease are provided. The formulas provided herein for dental and periodontal disease include those that are adapted for use by diabetics to provide additional benefits for the treatment or prevention of diabetic complications.

The compositions of this invention combine components which control oxidative stress, provide for appropriate neovascular regulation, provide factors necessary for stimulation or promotion of collagen synthesis and vascular tissue restoration, and preferably improve nutrient, e.g., mineral and vitamin, balance in an individual having conditions or symptoms associated with microangiopathy, particularly for those having diabetic complications and more particularly for those having diabetic retinopathy and/or nephropathy. Nutrients, vitamins and cofactors are provided at least in part to compensate for nutrient spillage that is typically observed in diabetes and the elderly. Preferred combinations of antioxidants and neovascular regulators include combinations of a plant extract providing antioxidant effect comprising bioflavanoids, e.g., proanthocyanidins, with a neovascular regulator selected from the group genistein, daidzein, soy isolate (a specific source of genistein and/or daidzein), cartilage or preferably chondroitin sulphate. A preferred neovascular regulator is chondroitin sulphate which also promotes or stimulates collagen synthesis and vascular tissue regeneration.

The multi-component compositions of this invention and treatment methods using them are based, at least in part, on a recognition that the conditions and symptoms associated with macroangiopathy and microangiopathy, are the result of a multi-factor etiology requiring consideration of multiple biochemical factors to successfully ameliorate or reverse these conditions or symptoms.

In more preferred compositions, the antioxidant, neovascular regulator, collagen synthesis factors, and nutrient components are combined with components that regulate glucose or insulin levels, regulate lipids, regulate cholesterol absorption, facilitate or enhance reconstruction of the vascular matrix and/or suppress inappropriate immune response.

In more preferred embodiments, the compositions of this invention employ different components having the same or similar biochemical or therapeutic functionality. These functionally similar components may differ in source (e.g., extracts of different plants), differ in chemical structure and/or different effective half-life on administration. Such combinations of different components with similar activities provide synergistic nonadditive benefits and improvements. Components of the compositions of this invention may themselves be multi-component mixtures with each subcomponent having differing functionality. Different composition components may have more than one biological function in the mixture and different components may have distinct, yet overlapping, biological functions. The use of functionally similar components which are structurally distinct or derived from different sources allows the inclusion of sufficiently high levels of total material to achieve a desired level of activity while avoiding the potential toxic effect that may result from use of high levels of any single component. The use of a combination of functionally similar components in a therapeutic/nutritional composition provides therapeutical active species having different half-lives. For example, preferred compositions of this invention combine two or more different antioxidants or components having antioxidant effect.

I. Specific Formulas for use in the Treatment and Prevention of Diabetic Complications Associated with Microangiopathy, such as Nephropathy and Retinopathy, Include:

1. Formula IA which comprises:

(i) A plant extract having antioxidant effect comprising bioflavanoids, particularly an extract providing a major source of proanthocyanidins, such as Bilberry extract, grape seed extract, or pine bark extract. Bioflavanoids of lower proanthocyanidin content, for example, ginkgo biloba, can also be used to supplement major sources; combinations of plant materials and extracts can also be employed;

(ii) Tea polyphenols providing for increased glucose tolerance and additional antioxidant benefit;

(iii) Absorbable zinc, preferably zinc(Krebs) to supplement dietary deficiency or loss due to diabetic excretion; and

(iv) A neovascular regulator selected from genistein and/or daidzein; soy isolate comprising genistein and/or daidzein; cartilage or chondroitin sulphate; chondroitin sulphate is a preferred neovascular regulator also associated with collagen synthesis; shark cartilage is a preferred cartilage preparation.

2. Formula IB which comprises:

Vitamin C;

Vitamin E;

Bilberry Extract (preferably low OPCs, e.g., 25% oligomers OPCs);

Pine Bark Extract (preferably high OPCs, e.g., 85% or greater OPCs);

Tea polyphenols;

Absorbable zinc, particularly zinc(Krebs);

Chondroitin sulphate; and

Soy isolate, or equivalent levels of genistein and/or daidzein; and optionally a cartilage preparation.

(OPCs are oligomeric proanthocyanidins)

3. Formula IC which comprises:

Formula IB; and

Glucosamine sulphate (a preferred glycosaminoglycan and source of glycosamine, a building block for collagen synthesis);

4. Formula ID which comprises:

Formula IC; and

Antioxidant carotenoids, such as lutien and/or zeaxanthin; and

Vitamin D3, preferably derivatives thereof which induce little or substantially no hypercalcification (e.g., 22-oxa-Vitamin D3).

5. Formula IE which comprises:

Formula ID;

Grape Seed Extract (also known as leucoanthocyanidin);

Vitamin A(acetate or palmitate);

A source of taurine, particularly homotaurine;

Absorbable magnesium, particularly magnesium (Krebs);

Absorbable calcium, particularly calcium (Krebs);

Absorbable chromium, particularly chromium picolinate; and

Absorbable potassium, particularly potassium citrate.

6. Formula IF which comprises:

Formula IE;

A source of essential fatty acids, particularly conjugated dienoic fatty acids; for example, linoleic acid

Folic acid;

Vitamin B2;

Vitamin B6; and

Vitamin B12.

7. Formula IG which comprises:

Formula IF; and

Melatonin.

8. Formula IH which comprises:

Formula IG;

Gymnema sylvestre;

Fenugreek Seed (preferably defatted powder);

A source of omega-3 fatty acids, particularly conjugated dienoic fatty acids, e.g., linoleic acid (ALA) and/or enosapentaenoic acid (EPA), a preferred source is ground flax seed;

Ginkgo biloba; and

Lycopene and/or beta-carotene (additional antioxidant carotenoids).

9. Formula IJ which comprises

Formula IH;

L-carnitine;

Quercitin;

Coenzyme Q, particularly Coenzyme Q₁₀(CoQ₁₀);

N-acetyl-L-cysteine; and

Thioctic acid (alpha lipoic acid).

10. Formula IK which comprises:

Formula IJ;

Absorbable selenium;

Indole-3-carbinol;

Glutathione;

Amino acids selected from: L-alanine, L-cysteine, or L-tryptophan;

Branched chain amino acids: L-leucine, L-isoleucine or L-valine;

Betaine hydrochloride;

pepsin; and

Sodium bicarbonate.

11. Formula IL which comprises:

Formula IK:

Eugenol; and

Pytosterols, particularly C24-substituted cholesterol derivatives xFormulas IA-IL are optionally combined with aspirin and NSAIDS (non-steroid anti-inflanmatories) and may optionally be combined with protamine sulphate and/or DHEA (dehydroepiandrosterone). Formulas IA-IK can be combined with the peptide hormones: calcitonin and /or amylin, which provide positive therapeutic benefit for individuals with diabetes. Red Wine Extract, a powerful proanthocyanidin-containing extract can also be employed in the Formulas IA-IL in place of, or in addition to, other proanthocyanidin

II. Specific Formulas for use in Wound Healing, Particularly Healing of Chronic, Persistent or Recurring Wounds including Decubitus Ulcers Include:

1. Formula IIA [Non-diabetic formula] which comprises:

(i) A plant extract having antioxidant effect comprising a major source of bioflavonoids, such as proanthocyanidins, including Bilberry extract, Grape seed extract or Pine Bark extract. Pine Bark extract is preferred. Pine Bark Extract is a superior antioxidant and anti-inflammatory which also promotes collagen synthesis and inhibits mammalian collagenases. Bioflavanoids of lower proanthocyanidin content, e.g., Ginkgo Biloba can also be used to supplement major sources; combinations of plant materials and extracts can also be employed;

(ii) A neovascular regulator particularly chondroitin sulphate which promotes rebuilding of collagenous tissue and enhances glucosamine performance;

(iii) Glucosamine sulphate and other sources of glucosamine which increase hyaluronic acid production and promote wound healing; and

(iv) A source of absorbable magnesium, preferably magnesium malate to support collagen synthesis and glucosamine utilization.

2. Formula IIB [Non-diabetic formula] which comprises

Pine bark extract;

Grape seed extract (leucoanthocyanidin);

Tea polyphenols;

Chondroitin sulphate;

Glucosamine sulphate;

Vitamin C (antioxidant which promotes collagen formation and strengthens capillaries);

Absorbable magnesium; and

Amino acids selected from: L-arginine, L-cysteine, glycine, L-methionine, L-threonine or L-proline

3. Formula IIC [Non-diabetic formula] which comprises

Formula IIB;

Thioctic acid (alpha-lipoic acid);

Bilberry extract;

Nicotinamide;

Aloe vera (preferably in powdered form);

Absorbable calcium, e.g., calcium citrate, calcium malate and mixtures thereof;

Vitamin A (antioxidant which increases collagen content of tissue);

Absorbable zinc, e.g., zinc (Krebs); and optionally

A cartilage preparation, particularly bovine cartilage.

4. Formula IID [Non-diabetic formula] which comprises:

Formula IIC;

A source of essential fatty acids, in particular, conjugated dienoic fatty acid, i.e., linoleic acid;

Folic acid;

Vitamin B12;

Vitamin B6;

Co-Q-10;

Vitamin D3 (derivatives having minimal hypercalcification);

Absorbable potassium, e.g., potassium citrate;

Vitamin K1; and

A source of taurine (L-taurine or homo-taurine).

5. Formula IIE [Non-diabetic formula] which comprises:

Formula IID;

Vitamin B2;

Vitamin B1;

Betaine hydrochloride;

Pepsin;

Sodium bicarbonate;

Ginkgo biloba;

Antioxidant carotenoids (Lutein or zeaxanthin or beta-carotene and/or lycopene); and

Vitamin B5 (pantothenic acid).

6. Formula IIF [Non-diabetic formula] which comprises:

Formula IIE;

N-acetyl-L-cysteine;

Protamine sulphate;

Soy isolate; and optionally,

Phytosterols, particularly C24 substituted cholesterol derivatives (e.g., Cholestatin III); and/or

Mineral complex (preferably without iron) including nutritional minerals not yet included in the formula.

7. Formula IIG [Non-diabetic formula] which comprises:

Formula IIF;

Vitamin B complex components (those not already in Formula IIF); and

A cartilage preparation, preferably bovine cartilage.

8. Any of Formulas IIA-IIG can be prepared as a diabetic formulation by including any of the following not already included:

Gymnema sylvestre;

Fenugreek seed;

Amylin;

Glutathione;

Thiotic acid;

Absorbable chromium, e.g., chromium picolinate; and

By deleting nicotinamide, if present.

9. Excess iron can be inhibitory to wound healing. Iron is thus excluded from the mineral complex of Formula IIF. Any of Formulas IIA-IIG, both the non-diabetic and diabetic formulations, can be prepared for use with iron-deficient individuals by addition of:

Absorbable iron sufficient to satisfy deficiency.

10. Omega-3-fatty acids are excluded from the wound healing compositions above as potentially inhibitory in the earlier stages of wound healing. These components can, however, be included in a preventative wound healing formula, before wounds occur such as when beginning a long hospital stay or after wound sites are sufficiently healed.

Formulas IIA-IIG, both non-diabetic and diabetic formulations, are intended for oral administration.

Any of the Formulas IIA-IIG (diabetic and non-diabetic) can be formulated as a wound healing ointment by addition of the following ingredients to the oral wound healing formulation:

(i) An antibiotic;

(ii) Honey (preferably raw) and/or sugar and/or glycerine;

(iii) An alginate (a gelling polysaccharide, preferably from seaweed, e.g., sodium or calcium alginate)

(iv) One or more amino acids selected from the group L-proline; L-cysteine; L-arginine; Glycine; L-threonine; or Branched chain amino acids (if not already included in oral formulation).

Any antibiotic appropriate for topical application can be employed including, for example, hydrogen peroxide (30%), polyethylene glycol 400, acetic acid, or betadine. Sugars can include brown sugar, caster sugar or powdered sugar. Wound healing ointments optionally include cartilage, allantoin and/or urea for additional wound healing benefit. Antibiotics and other active ingredients are included in wound healing ointment in an amount effective for providing the desired therapeutic or nutrient effect (e.g., to compensate for a local deficiency). Sugars, honey or glycerine can be replaced with a pharmaceutical carrier appropriate for ointment formulation. In preferred embodiments, sugars and honey (or pharmaceutical carrier) represent about 50% to about 70% (be weight); antibiotics represent 20-40% (by weight); and other ingredients represent about 1-20% (by weight) of the ointment.

Wound healing ointments can also contain pH control agents, vitamins and/or mineral combination, additional vascular enhancers, osmotic stabilizers, and enzymes.

Excipients for topical application include among others: alginate, pectin, gelatin, gelatin derivatives, cellulose derivatives, quar gum, acacia gum, karaya gum, tragacanth gum, locust bean gum, agar, dextran, derivatives of dextran, ghatti gum, xanthan gum, polyvinylpyrolidone, polyethylene, polyethylene glycol, glycerol, polypropylene glycol.

Other additives that may be combined with ointments and other topical formulas include coloring agents, flavoring agents, thickeners, emulsifying agents, surfactants, and solubilizing agents.

Formulas IIA-IIH are optionally combined with aspirin and or NSAIDS where appropriate. Red Wine Extract, a powerful proanthocyanidin-containing extract can also be employed in the Formulas IIA-IIH in place of, or in addition to, other proanthocyanidin.

Formulas IIA-IIH (diabetic and non-diabetic) can optionally include:

Dragon's Blood (a proanthocyanidin containing extract with particular wound healing function); and/or

Centella asiatica or its extract.

III. Specific Formulas for use in the Treatment and Prevention of Neuropathy Include:

1. Formula IIIA [Non-diabetic] which comprises:

(i) A plant extract having antioxidant effect comprising a major source of proanthocyanidins, such as Bilberry extract, grape seed extract or pine bark extract. Bioflavanoids of lower proanthocyanidin content, e.g., ginkgo biloba can also be used to supplement major sources; combinations of plant materials and extracts can also be employed;

(ii) A neovascular regulator, particularly chondroitin sulphate; and

(iii) Glucosamine sulphate (a source of glucosamine).

2. Formula IIIB [Non-diabetic] which comprises:

Pine Bark extract;

Chondroitin sulphate;

Glucosamine sulphate;

Absorbable magnesium, e.g., magnesium malate;

Absorbable calcium, e.g., calcium (Krebs);

Thioctic acid (alpha-lipoic acid);

Ginkgo biloba;

Tea polyphenols;

Vitamin C; and

A source of essential fatty acids. (Vitamin C and essential fatty acid may both be supplied as ascorbyl-gamma-linoleic acid, for example.

3. Formula IIIC [Non-diabetic] which comprises:

Formula IIB;

Vitamin B complex;

Co-Q-10;

Vitamin E;

Vitamin D3, preferably a derivative inducing little or substantially no hypercalcification;

Vitamin K; and

A source of omega-3-fatty acids, e.g., flax seed.

4. Formula IIID [Non-diabetic] which comprises:

Formula ITIC;

Absorbable potassium, e.g., potassium citrate;

Absorbable zinc, e.g., zinc (Krebs);

Soy isolate;

Antioxidant carotenoids (e.g., lutien or zeaxanthin or beta carotene and/or lycopene); and

Folic acid.

5. Formula IIIE [Non-diabetic] which comprises:

Formula IIID;

Grape seed extract (leucoanthocyanidin);

Vitamin A;

A source of taurine (e.g., homotaurine or L-taurine); and

Protamine sulphate.

6. Formula IIIE [Non-diabetic] which comprises:

Formula IIID; and/or

Branched-chain amino acids; and/or Melatonin; and/or

A source of cartilage or a cartilage preparation, e.g., shark cartilage.

7. Formula IIIF [Non-diabetic] which comprises:

Formula IIID±options of Formula IIIE;

Absorbable selenium;

N-acetyl-L-cysteine;

Glutathione;

Betaine hydrochloride;

Pepsin;

Sodium bicarbonate;

Bilberry extract; and optionally Phytosterols; and/or

Mineral complex (except for the minerals noted above in Formula IIIA-E).

8. Formulas IIIA-IIIF can be prepared as a diabetic formulation by addition of any of the following not already included:

Gymnema sylvestre;

Fenugreek seed;

Glutathione;

Thioctic acid (alpha-lipoic acid, if not already included in formula);

Absorbable chromium, as chromium picolinate; and optionally, Myo-inositol and biotin.

Formulas IIIA-IIIF for treatment and prevention of neuropathy (diabetic and non-diabetic) can be combined with aspirin and/or NSAIDS.

Formulas IIIA-IIIF (diabetic and non-diabetic) can also include glutathione peroxidase which has additional antioxidant effect. Red Wine Extract, a powerful proanthocyanidin-containing extract can also be employed in the Formulas IIIA-IIIF in place of, or in addition to, other proanthocyanidins.

Components of Formulas IIIA-IIIF (diabetic and non-diabetic) can be formulated in appropriate carrier materials for topical application to affected areas.

IV. Specific Formulas for use in the Prevention and Treatment of Cardiovascular Disease Include:

1. Formula IVA [Non-diabetic] which comprises:

(i) A plant extract having antioxidant effect comprising bioflavanoids, particularly an extract providing a major source of proanthocyanidins, such as Bilberry Extract, Grape Seed Extract, or Pine Bark Extract. Bioflavanoids of lower proanthocyanidin content, for example, Ginkgo Biloba, can also be used to supplement major sources; combinations of plant materials and extracts can also be employed;

(ii) Absorbable zinc, preferably zinc(Krebs) to supplement dietary deficiency or loss due to diabetic excretion; and

(iii) A neovascular regulator selected from genistein and/or diadzein; soy isolate comprising genistein and/or diadzein; shark cartilage or chondroitin sulphate.

2. Formula IVB [Non-diabetic] which comprises:

Vitamin C;

Vitamin E;

Bilberry Extract (preferably low OPCs, e.g., 25% oligomers OPCs);

Pine Bark Extract (preferably high OPCs, e.g., 85% or greater OPCs);

Tea polyphenols;

Absorbable zinc, particularly zinc(Krebs);

Soy isolate, or equivalent levels of genistein and/or diadzein; and

Chondroitin sulphate;

Glucosamine sulphate; and optionally a cartilage preparation, e.g., shark cartilage

(OPCs are oligomeric proanthocyanidines)

3. Formula IVC [Non-diabetic] which comprises:

Formula IVB;

Antioxidant carotinoids, such as lutein and/or zeaxanthin;

Grape Seed Extract (also known as leucoanthocyanidin);

Vitamin A (acetate of palmitate);

A source of taurine, particularly homotaurine;

Protamine sulphate;

Absorbable magnesium, particularly malate and/or magnesium (Krebs);

Absorbable calcium, particularly calcium (Krebs);

Absorbable potassium;

Vitamin K1, an anti-atherosclerotic and antioxidant: and

Vitamin D3, preferably derivatives thereof which induce little or substantially no hypercalcification (e.g., 22-oxa-Vitamin D3).

4. Formula IVD which comprises:

Formula IVC

A source of essential fatty acids, e.g., conjugated dienoic fatty acids, such as linoleic acid;

Melatonin;

Folic acid;

Vitamin B2;

Vitamin B6;

Vitamin B12

Antioxidant carotenoids, including lycopene and/or beta carotene; and

A source of omega-3-fatty acids, e.g., flax seed.

5. Formula IVE [Non-diabetic] which comprises:

Formula IVD;

Ginkgo Biloba; and

Quercitin (or other antioxidant bioflavonoid)

6. Formula IVF [Non-diabetic] which comprises:

Formula IVE;

Coenzyme Q, particularly Coenzyme Q₁₀ (CoQ10);

N-acetyl-L-cysteine;

Glutathione;

Thioctic acid (alpha lipoic acid);

Absorbable selenium (an organoselenium compound, such as selenomethionine);

Indole-3-carbinol;

Glutathione;

Betaine hydrochloride;

Pepsin;

Sodium bicarbonate;

Nicotinamide;

Amino acids selected from: L-arginine, glycine, L-methionine, L-tyrosine, L-tryptophan, or gamma-amino butyric acid; and

Phytosterols, particularly C-24-substituted cholesterol.

7. Formulas IVA-IVF can be prepared as a diabetic formulation by addition of any of the following not already included:

Gymnema sylvestre;

Fenugreek seed;

Glutathione;

Thioctic acid;

Absorbable chromium, e.g., chromium picolinate; and by deletion of nicotinamide, if present.

The compositions. of formulas IVA-IVF (diabetic and non-diabetic) can be combined with aspirin and/or NSAIDS. Red Wine Extract, a powerful proanthocyanidin-containing extract can also be employed in the Formulas IVA-IVF in place of, or in addition to, other proanthocyanidin

V. Specific Formulas for use in the Prevention and Treatment of Dental Caries and Periodontal Disease Include:

1. Formula VA [Non-diabetic] which comprises:

(i) A plant extract having antioxidant effect comprising a major source of proanthocyanidins, such as Bilberry Extract, Grape Seed Extract, or Pine Bark Extract. Bioflavanoids of lower proanthocyanidin content, for example, Ginkgo biloba, can also be used to supplement major sources; combinations of plant materials and extracts can also be employed;

(ii) Absorbable calcium, such as calcium citrate, calcium malate or mixtures thereof; and

(iii) A source of Vitamin D3, preferably a Vitamin D3 derivative or analog that induces little or substantially no hypercalcification.

2. Formula VB [Non-diabetic] which comprises:

Pine bark extract;

Tea polyphenols;

Absorbable calcium, preferably calcium citrate/malate; and

Vitamin D3, preferably derivatives thereof which induce little or substantially no hypercalcification (e.g., 22-oxa-Vitamin D3).

3. Formula VC [Non-diabetic] which comprises:

Formula VB;

Absorbable magnesium, particularly magnesium malate;

Absorbable strontium;

L-lysine;

Absorbable zinc, e.g., zinc (Krebs); and

N-acetyl-L-cysteine.

4. Formula VD [Non-diabetic] which comprises:

Formula VC;

Cysteine;

Absorbable silicon (as a silicate, e.g., as a trisillicate salt);

Chondroitin sulphate;

Glucosamine sulphate;

Quercitin (or other antioxidant bioflavonoid);

Absorbable potassium; and

Vitamin C.

5. Formula VE [Non-diabetic] which comprises:

Formula VD;

Absorbable manganese, particularly manganese aspartate;

Soy isolate;

Vitamin K1 (a regulator of calcium metabolism);

Vitamin A;

Thioctic acid (alpha lipoic acid);

Co-Q-10; and optionally

A cartilage preparation, preferably bovine cartilage.

6. Formula VF [Non-diabetic] which comprises:

Formula VE;

Absorbable cadmium;

Betaine hydrochloride;

Pepsin; and

Sodium bicarbonate.

7. Formula VG [Non-diabetic] which comprises:

Formula VF;

Vitamin E;

Omega-3-fatty acid source, e.g., flax seed;

Grape seed extract (leucoanthocyanidin);

Bilberry extract; and optionally sulphated saccharides (e.g., sucraflate);

8. Formula VH [Non-diabetic] which comprises:

Formula VG;

L-taurine;

Folic acid;

Glutathione;

A source of essential fatty acid;

Ginkgo biloba;

Protamine sulphate;

Vitamin B complex; and optionally

Plant sterols.

9. Formulas VA-VH can be prepared as a diabetic formulation by addition of any of the following not already included:

Gymnema sylvestre;

Fenugreek Seed;

Glutathione;

Thioctic acid; and

Absorbable chromium (e.g., chromium picolinate).

Compositions of Formulas VA-VH (diabetic and non-diabetic can be combined with aspirin and/or NSAIDS, if appropriate. Red Wine Extract, a powerful proanthocyanidin-containing extract can also be employed in the Formulas VA-VH in asplace of, or in addition to, other proanthocyanidin.

The components listed in all formulas above, are believed to have the biological nutrient or therapeutic functions as listed above and as indicated in Tables 1 and 2, where a single component may provide multiple functions.

Compositions of the present invention also include those in which the primary compositions, Formulas IA-VA, are combined with any of the additional ingredients of other specific formulas IB-IK, IIB-IIG, IIIB-IIIF, IVB-IVF, VB-VH, respectively, of its type.

Formulas of this invention listed above can also be combined with garlic extract (allicin), licorice extract, ginger, red wine extract, citrus pectin and/or marine tunicates or their isolates each of which can function for neovascular regulation and may provide additional therapeutic or nutritive benefit. The formulas of this invention can optionally include nutrients, vitamins and minerals other than those specifically listed to supplement particular nutritional deficiencies of given individuals, for example, chromium, iron, or other mineral may be provided or its concentration increased to supplement a given deficiency. Similarly, a particular vitamin or amino acid deficiency can be supplemented. Analogously, a given formulation can be adapted for sensitivities or allergies of a given individual.

Components that enhance or facilitate desirable enzyme activity, e.g. lysyl oxidase (an enzyme which participates in collagen synthesis); nitric oxide inhibitors, other antioxidant carotenoids or flavanoids, additional antihyperlipoproteinemics, including probucol and blood thinning agents, e.g. heparin can be combined with any of the formulas listed above.

Celluar antioxidants, such as the enzymes: superoxide dismutase and catalyze or thiols, including glutathione peroxidase, can be included in any of specific formulas listed above. L-carnitine (which may be in the form of L-acetyl carnitine or L-propionyl carnitine) can be combined with any of the specific formulas above.

Treatment using the compositions of this invention can be combined with hormone therapy and or hormone supplementation, including estrogenic hormone therapy or supplementation, thyroid hormone therapy or supplementation, treatment or supplementation with human growth hormone (HGH) and/or treatment or supplementation with DHEA (dehydroepiandrosterol).

The formulas of this invention can also be combined with appropriate growth factors, growth factor inhibitors and growth factor binding agents including, among others, fibroblast, epidermal, interleuken transforming and platelet-derived growth factors, agents that bind hyaluronic acid and/or collagen. The formulas of this invention can also be combined with immune suppression of T-lymphocytes.

The formulas of this invention can also be employed in combination with therapeutic methods shown to have beneficial effect for the disorders, conditions and diseases discussed herein. For example, wound healing formulas (oral and topical) can be used in combination with oxygenation therapy for improved wound healing benefit.

Other optional components of the formulas of this invention include antioxidants and/or preservatives, such as BHT (Butylated hydroxytoluene), BHA (Butylated hydroxyanisole), ethoxiquin and diphenyl phenylenediamine.

In general the amount of each component employed in the different compositions of this invention is sufficient to provide the desired therapeutic effect(s) or nutritive effect(s), as listed in Tables 1 and 2 and discussed herein, to an individual and avoid toxicity with continuing regular dosing. Because compositions of this invention can have multiple components with similar functionality, the effective amount of any given component needed to provide a given level of function in a given composition will depend on the quantities of other functionally similar components to be included in the composition.

Table 3 provides a list of preferred components for the compositions of this invention providing a preferred range of amounts of individual components that can be combined in the formulas of this invention. The amounts listed in Table 3 are average daily adult dosages.

Table 4 provides a list of preferred components for a therapeutic and preventative composition for diabetic complications, e.g., retinopathy and nephropathy of this invention. The table provides a preferred range of amounts of individual components that are combined in the formulas of this invention. The amounts listed in Table 4 are average daily adult dosages. In Table 4, two preferred diabetic complications formulas are provide. Formula B has somewhat higher levels of folic acid, riboflavin and pyridoxine compared to formula A. (Formula B employs the palmitate form of Vitamin A, while formula A employs Vitamin A acetate.) The specific compositions (A and B) of Table 4 are intended as an initial treatment dose. Lower daily dosage compositions can be employed after initial treatment to maintain beneficial effects. Alternatively, lower daily dosage compositions can be employed to forestall or prevent diabetes-related conditions in those at risk for developing them. Preventative and maintenance compositions may contain ingredients in addition to those listed in Table 1. Variation of the amounts of individual components in the preferred composition by up to about +/−20% will not significantly affect nutritive or therapeutic value. A broad range of effective amounts for each preferred component is provided in Table 3.

The primary formulas of this invention useful for treatment of symptoms and conditions associated with microangiopathy and macroangiopathy comprise components that (1) have antioxidant function to control oxidative stress, (2) are neovascular regulators which control angiogenesis, (3) promote and/or stimulate collagen synthesis and (4) optionally stabilize glucose and/or amylase factors; or (5) optionally supplement dietary deficiencies and counteract non-utilization or spillage by diabetics. Table 1 provides a summary of the biochemical functions of components that are useful in combination with the components of those primary formulas. A single component may provide more than one of the listed biological functions in a given composition.

One or more of the functionalities listed in Table 1 can be provided in the compositions of this invention by art-known drug equivalents. For example, art-known antidiabetic agents, antihypertensives, angiotensin converting enzyme inhibitors, vasodilators, anticholesteremics, antihyperlipoproteinemics, angiogenesis regulators, and enzyme co-factors can be combined in effective amounts for ameliorating symptoms and conditions associated with microangiopathy, particularly retinopathy and nephropathy, with formulas of this invention.

Compositions of this invention can be provided in a variety of nutrient and dosage forms including pills, tablets, capsules, lozenges, powders, solutions, suspensions, injection dosage forms and the like. Compositions of this invention can be administered to individuals orally, intravenously, and by various forms of injection and various forms of absorption (e.g., sublingual). Active ingredients of the formulas of this invention can be combined with excipients, fillers, buffering agents and the like to prepare desired dosage forms. Generally preferred dosage forms are those appropriate for oral administration. Wound healing compositions and compositions for treatment of neuropathy are provided for topical application.

This invention also encompasses methods of treatment to ameliorate the symptoms and disease conditions associated with microangiopathy and macroangiopathy which comprise administration of the compositions of this invention to an individual suffering from symptoms or conditions resulting these disorders. More specifically, the invention provides methods for ameliorating diabetic retinopathy and nephropathy. Methods of this invention can be combined with other compatible known methods for treatment of diabetic complications. The compositions of this invention for treatment of diabetic complications are best applied in a treatment regime that emphasizes good diabetes control. Methods of this invention can also ameliorate ocular conditions including macular degeneration, glaucoma and cataracts.

### DETAILED DESCRIPTION OF THE INVENTION

The nutrient and therapeutic compositions of this invention are generally directed toward the improvement of disease conditions and symptoms that are associated with vascular and capillary degeneration: macroangiopathy and microangiopathy. Compositions of this invention also provide for prevention or retardation of the development or worsening of certain disease conditions or symptoms associated with vascular and capillary degeneration in individuals at risk for developing these disorders, for example, in individuals with diabetes or individuals exhibiting symptoms of cardiovascular disease. This invention provides formulas for treatment and prevention of diabetic complications including retinopathy, neuropathy and nephropathy. Formulas of this invention are also useful in the treatment and prevention of non-diabetic retinopathy, neuropathy and nephropathy. Formulas of this invention are also useful in the prevention and treatment of the symptoms and disease conditions of cardiovascular disease. Formulas of this invention are useful in wound treatment and are particularly useful in treating recurrent or slow-to-heal wounds including those that are a complication of diabetes. Formulas of this invention are also useful in the prevention and treatment of dental and periodontal disease conditions.

The formulas of this invention that are useful in the treatment and prevention of the various disease conditions discussed above combine a number of related ingredients. The therapeutic and preventative compositions of this invention are based at least in part on the inventor's recognition of similarities in etiology of the various disease conditions discussed above. In particular, the inventor considers that these conditions and disorders are, at least in part, caused by or exacerbated by oxidative stress and tissue destruction associated with oxidative damage. Further, the inventor considers that the disorders discussed above are, at least in part, caused by or exacerbated by microangiopathy and/or macroangiopathy, i.e., vascular and capillary degeneration. Vascular and capillary degeneration is, at least in part, caused by antioxidant stress. Further, the inventor considers that in each of the disease conditions and symptoms, for which formulas are provided herein, that stimulating and or promoting collagen synthesis is an important factor in prevention and treatment. In this regard, the various disease conditions discussed herein also relate in part aberrant tissue growth, for example due to lack of proper growth factors or lack of growth factor inhibitors. Furthermore, conditions associated with microangiopathy also suffer from the effects of deprivation of adequate nutrient, vitamin, cofactor and mineral supplies and particularly from inadequate supplies of nutrients, cofactors and the building blocks needed for restoration of the collagen matrix which is necessary for regeneration and healing of vascular tissue and tissue in general.

Diabetic complications of retinopathy and nephropathy are clearly associated with microangiopathy, improperly controlled vascularization and concomitant weakening of capillaries. The formulas of this invention for treatment of diabetic complications include antioxidants, neovascular regulators (particularly angiogenesis regulators) and factors that promote or stimulate collagen synthesis and restoration of the collagen matrix.

Cardiovascular disease is directly linked to vascular degeneration. Tissue damage induced, at least in part, by oxidative stress provides sites for lesion formation and plaque accumulation. Formulas of this invention for use in treatment and prevention of cardiovascular disease include antioxidants to prevent or limit oxidative tissue damage, growth factors (neovascular regulators) that stimulate repair of vascular tissue, factors that stimulate or promote collagen synthesis and other components of benefit for cardiovascular disease. The cardiovascular compositions of this invention can be formulated to include ingredients that are beneficial for diabetics.

The wound healing compositions of this invention are based on the premise that wounds that resist healing part from infection, result, at least in part, from microangiopathy. As noted above, microganiopathy is believed to involve oxidative stress, deficient neovascular regulation and deficient collagen synthesis. Microangiopathy is believed to promote nutrient and oxygen deprivation, and ineffective immune response at the wound site. All of these factors: oxidative stress, deficient neovascular regulation, deficient collagen synthesis, nutrient and oxygen deprivation and local immune deficiency are believed to contribute and/or exacerbate the slow healing process. All of these factors would contribute to destruction of cells and tissue faster than they can be replaced, leading to wounds that do not heal or that worsen.

The wound healing compositions of this invention concurrently attenuate these factors by (1) controlling oxidative stress and providing protection from free-radicals and other biological oxidation agents, (2) providing neovascular regulators, particularly inhibitors of angiogenesis, and/or collagen factors which promote or stimulate collagen synthesis and/or inhibitors of mammalian collagenases to enhance capillary and tissue repair, and (3) compensating for inadequate nutrient delivery by supplying minerals, vitamins and amino acids. The wounding healing compositions of this invention also provide for immune inflammation. The wounding healing compositions of this invention can be formulated to include ingredients that are beneficial for diabetics.

The compositions of this invention for treatment of neuropathy are based on the premise that neuropathy results, at least in part, from microangiopathy. As noted above, microangiopathy is believed to involve oxidative stress, immune inflammation, deficient neovascular regulation and deficient collagen synthesis. Oxidative stress, deficient neovascular regulation, deficient collagen synthesis, nutrient and oxygen deprivation and local immune deficiency are believed to contribute and/or exacerbate the slow healing process. All of these factors would contribute to destruction of cells and tissue faster than they can be replaced, leading to nerve tissue damage. In addition to providing for antioxidants, growth factors, factors that promote tissue growth and nutrient balance, formulas of this invention for neuropathy also provide additional vitamins, minerals and cofactors linked to improvement in neuropathy. Neuropathy is a significant complication of diabetes. The neuropathy compositions of this invention can be formulated to include ingredients that are beneficial for diabetics.

The neuropathy compositions of this invention concurrently attenuate these factors by (1) controlling oxidative stress andimmune inflammation and providing protection from free-radicals and other biological oxidation agents, (2) providing neovascular regulators, particularly inhibitors of angiogenesis, and/or collagen factors which promote or stimulate collagen synthesis and/or inhibitors of mammalian collagenases to enhance capillary and tissue repair, and (3) compensating for inadequate nutrient delivery by supplying minerals, vitamins and amino acids. The neuropathy compositions of this invention can be formulated to include ingredients that are beneficial for diabetics.

The inventor has discovered that there is a significant improvement in periodontal disease and gingivitis in individuals who regularly take antioxidant supplements. Thus, oxidative stress is believed to be a factor in the development of such disease. It is believed that there is an indirect relationship between microangiopathy and dental and gum disease including periodontal disease. Gingivitis is associated with bacterial infection, however, the local environment and condition of the teeth, bone and gum tissue is believed to be important in development of dental and gum disease and infection. Tissue damage is believed to allow and exacerbate infection. Microangiopathy is also believed to also cause tissue damage resulting in nutrient and oxygen deficiency and exacerbation of tissue damage. Formulas of this invention for treatment and prevention of dental and gum disorders include antioxidants, factors that stimulate tissue repair and collagen synthesis and other nutrient and vitamin components that have benefit for the condition of the teeth and gums. Gum disease and tooth loss are complications of diabetes. The dental and periodontal compositions of this invention can be formulated to include ingredients that are beneficial for diabetics.

The treatment methods described herein employing the formulations of this invention are believed to derive unique and unexpected benefits from complementary and synergistic interactions between the various formula components acting together upon the various symptoms and conditions associated with the various diseases and disorders discussed herein. The success of these compositions in the treatments described is, at least in part, attributable to the multi-factor strategy employed to balance nutrient and metabolic deficiencies and to control oxidative stress, while promoting or stimulating vascular healing and/or collagen matrix repair, and inhibiting angiogenesis.

A description of various components (and their functional equivalents) of the formulas of the present invention follows:

Antioxidants

Antioxidants and antioxidant precursors are included in the compositions of this invention to combat oxidative stress and slow the deterioration of collagen tissues. In general, antioxidants are believed to protect vascular and capillary tissue to ameliorate macroangiopathy and microangiopathy. In the more preferred compositions of this invention a complementary antioxidant strategy is employed. Different chemical types of antioxidants are combined to provide enhanced antioxidant effect. Preferred antioxidant combinations include both hydrophilic (having affinity for water or polar groups) and hydrophobic (having an affinity for lipids) antioxidants and combinations of antioxidants from different natural plant sources. In a preferred embodiment, antioxidant vitamins (vitamins C or E), the mineral zinc and different plant bioflavonoid sources are combined to achieve complementary and synergistic antioxidant effects related to microvascular protection and healing associated with diabetic complications. In addition, antioxidant bioflavanoids, such as quercitin, and antioxidant carotenoids, such as lycopene, can be included for additional antioxidant effect.

Vitamin C or ascorbic acid can be provided in compositions of this invention in a variety of forms. Vitamin C is available from a variety of natural sources, which may also be employed in the compositions of this invention. Vitamin C is a hydrophilic antioxidant generally found in hydrophilic environments in the body, i.e., the bloodstream, the eye, interstitial spaces between cells and within cell membranes. It not only functions as a scavenger for singlet oxygen and hydroxy radicals, but it also replenishes spent Vitamin E by replacing electrons. In the bloodstream, Vitamin C reduces platelet aggregation, an anti-sclerotic effect. Vitamin C has a short half life and may interfere with diabetic glucose testing. For these reasons, it may be desirable, particularly in formulas for treatment of diabetic complications, to provide Vitamin C in smaller, more frequent doses or in a time released form. Forms of vitamin C suitable for use in the formulas of this invention include ascorbic acid, calcium and/or sodium ascorbate, and nicotinamide ascorbate.

Indole-3-carbinol is an antioxidant that provides functions similar to that provided by Vitamin C, however, is considered to provide protection against a broader range of biological oxidation agents.

Tocopherols (Vitamin E, d-alpha-tocopheryl salts) are hydrophobic, lipid-based compounds with antioxidant function. They are believed to have a primary role in protecting cell membranes from lipid peroxidation. Tocopherols also scavenge free radicals in the blood and help to protect Vitamin A and selenium. D-alpha tocopherol forms, the natural forms of Vitamin E, are preferred over the less bioactive d,1-tocopherol forms. Tocopherols can be provided in a variety of forms with different counterions. D-alpha-tocopheryl acetate and gamma-tocoperol are preferred for use in the compositions of this invention. Because some subjects can exhibit a slight rise in blood pressure when Vitamin E is first taken, smaller more frequent doses or a time-released form of Vitamin E may be more appropriate for microvascular protection in diabetics.

Lutien also called xanthophyll, a carotinoid related to beta-carotene, but not a pro-Vitamin A carotinoid, is itself a lipid peroxide scavenger and appears to promote the production of zeaxanthin, another abundant and powerful lipid-based antioxidant. Lutien is found in the human retina and is believed to act, possibly in a complementary manner with zinc, to protect retinal and macular tissue from oxidative damage. Lutien and zeaxanthin appear to perform the vast majority of the antioxidant function in the lens, retina and macula, of the eye with their highest concentrations found in the macula. Lutien and zeaxanthin form the yellow pigment in the macula and central area of the retina which absorbs blue light and thereby appears to prevent photic damage to the macula. Lutein is reported to be deficient in the eyes of those having age-related macular degeneration. Zeaxanthin, an isomer of lutein, isolated from yellow corn grits, can be employed in compositions of this invention in place of or in addition to lutien.

Beta-carotene is an optional component of the compositions of this invention. It is a lipid-based, pro-vitamin A antioxidant which quenches singlet oxygen and scavenges free radicals. It plays a role in protecting against lipid peroxidation and this function is especially valuable in the retina which contains high levels of poly-unsaturated fatty acids. Beta-carotene may also have a synergistic effect with other carotenoids, including lutein or zeaxanthin, for enhanced antioxidant function. In preferred antioxidant combinations, two or more carotinoid antioxidants are combined. Lycopene is another antioxidant flavanooid. Antioxidant flavanoids, including among others the flavanone glycosides quercitin, naringin, rutin and their aglucons, are superoxide scavengers and inhibit oxidation of LDL. In preferred antioxidant combinations, two or more antioxidant flavanoids are combined.

Alpha-lipoic acid (thioctic acid), which can be provided in the acid form or as an appropriate lipoate salt, e.g., sodium lipoate, is an antioxidant and free radical scavenger that reacts with reactive oxygen species including superoxide, hydroxyl radical, hypochlorous acid, peroxy radical, and singlet oxygen. Its reduced form, dihydrolipoate, is also an effective antioxidant. The d-form is the naturally-occurring optical isomer and preferred. The dl-form is available and can be employed in place of the d-form. Alpha-lipoic acid and its reduced dihydrolipoate form can bind to proteins including albumin which can prevent glycation reaction.

Creatine phosphate is reported to have an anti-ischemic effect and to function as an anti-oxidant. It may also function to protect myocardial tissue from damage due to free radiacals.

The mineral zinc, which is discussed in more detail below, is associated with protecting against lipid peroxidation in retinal tissue, possible due to its enhancement of superoxide dismutase function. The mineral potassium, also discussed below, inhibits superoxide anion.

Bioflavonoids containing proanthocyanidins scavenge free radicals and chelate some minerals to prevent them from oxidizing. These bioflavonoids are found in most plants from which they can be extracted. Commercially available proanthocyanidin-containing plant extracts include: grape seed extract (also called leucoanthocyanidin), pine bark extract (including “Pycnogenol” (Trademark, Horphag)), and Bilberry extract. Ginkgo Biloba and other plants can provide bioflavonoids of lower proanthocyanidin content which can also supplement antioxidant effect. These materials and extracts contain rather complex mixtures of catechins, tannins, oligomers and proanthocyanidins, at least some of which protect membranes from lipid peroxidation, and inhibit superoxides. They are hydrophilic antioxidants, which are many times more effective than most antioxidant nutrients at controlling free radicals, superoxides and lipid peroxides. Individual plant materials which can provide proanthocyanidins may also provide other therapeutic benefits, for example, garlic and willow bark (a source of salicylic acid) may provide additional benefit.

Oligomeric proanthocyanidins (OPCs) are polymer chains of 10 or less catechins which yield red anthocyanidin when boiled in an aqueous solution of 10% hydrochloric acid. Proanthocyanidins do not contain condensed tannins but are composed of nearly 60% catechin forms which have an extremely high affinity for collagen. Catechin binds tightly to collagen, modifies its structure by crosslinking and causes it to be resistant to enzyme degradation, such as by collagenase, or by lipid peroxidation and superoxide radicals. Proanthocyanidins inhibit capillary resistance and capillary permeability and, thus, improve vascular damage and deterioration. Collagen accumulates in vessel walls in endothelia, the connective matrix, elastin and phospholipids which helps to maintain structural integrity and protect these structures from peroxide anion damage. Plant extracts employed in this invention as sources for proanthocyanidins contain varying levels of OPCs. Antioxidant effectiveness of an extract generally increases with increasing levels of OPCs in the extract.

Dragon's Blood Croton spp. (Pieters, L., et al. (1995) Phytomedicine 1: 17-22) comprising antioxidant proanthocyanidines, has been associated with wound healing. This material can be optionally combined with wound healing compositions of this invention.

Red wine extract is a source of proanthocyanidins and tannins. Such extracts have anti-oxidant effect and may function to prevent platelet aggregation.

Catechins normally protect cell membranes from lipid peroxidation. Proanthocyanidins also help to deliver and bind Vitamin C to cell cites and can function to replace Vitamin C at times of ascorbic acid deprivation.

Compositions of this invention can contain one or more sources of proanthocyanidins which are included as antioxidants in the formula. Proanthocyanidins also promote vascular healing and integrity by restoring the collagen matrix. Different sources of proanthocyanidins, i.e., plant extracts, can also display other therapeutically beneficial functions in compositions of this invention.

Bilberry extract is useful in the treatment of retinopathy. It may contain 5 types of anthocyanocides which account for most of its activity and 25% of its volume. While Bilberry extract inhibits superoxides and lipid peroxide to some degree, it is low in oligomeric proanthocyanidins (OPCs) and therefore is less effective at controlling these free radical forms than leucoanthocyanidin (grape seed extract, for example) described below. Bilberry has an unusual anti-inflammatory effect, possibly because it can suppress leukotriene production. In addition, proanthocyanidins can achieve concentrations in tissue (kidney and skin) up to 5 times the level contained in the bloodstream. High tissue concentrations can remain up to 24 hours after serum concentrations have been depleted. These factors contribute to Bilberry's role in microvascular protection and repair and are particularly relevant to nephropathy, but also useful in treating other diabetic complications described herein.

The proanthocyanidin-containing extract of grape seeds includes the material called leucoanthocyanidin. This commercially available material is obtained from white grape pips and is the most effective form of proanthocyanidin, yet discovered, for inhibiting superoxides and lipid peroxidation. This is believed to be due to the high level of oligomeric proanthocyanidins (OPCs) in the grape seed extract which strongly relates to vascular stabilization as described above. Red grape extract which is a good source of resveratrol can also be employed in this invention for antioxidant effect and other benefits.

Pine Bark Extract, some preparations of which are known by the trade name “Pycnogenol,” is similar to leucoanthocyanidin, having relatively high OPC levels, but may possess better ability to suppress phagocytes.

Ginkgo biloba is a “middle range” proanthocyanidin possessing many of the functional characteristics of both Bilberry extract and grape seed extract, but these active components are apparently present in lower concentrations. Ginkgo biloba can cause dilation of arteries, capillaries and veins and inhibit platelet aggregation. Ginkgo biloba also functions to inhibit high blood pressure which is an important reason for its inclusion in compositions of this invention.

Green tea extract, tea polyphenols, contains a small amount of 2-3% of proanthocyanidin. It nevertheless is a potent antioxidant for lipid peroxides, superoxides and hydroxyl radicals. It contains relatively high concentrations of (−) epigallocatechin gallate (EGCg), a condensed tannin polyphenol. In addition to antioxidant function, tea polyphenols also have anti-platelet, anti-cholesterolemia, anti-hypertension, anti-hyperglycemic and anti-mutagenic activities. Tea polyphenols also assist theoflavin digallate in acting as an angiotensin converting enzyme inhibitor, but do not have the undesired pro-oxidant properties of captopril.

The five sources of bioflavonoids, Bilberry, grape seed extract (leucoanthocyanidin), Ginkgo biloba, pine bark extract (“Pycnogenol”) and green tea extract (tea polyphenols) described above have significant complementary and synergistic chemical function that in combination with other ingredients and antioxidants in the formulas of this invention promote the microvascular benefits needed to improve retinopathy as well as other diabetic complications.

N-Acetyl-l-cysteine is a free radical scavenger and is very effective for lowering lipoprotein (a) [LP(a)] concentrations in vivo. High levels of LP(a) are associated with increased risk to atherosclerosis and thrombic disease and are believed to accelerate microvascular disease in diabetes. Glutathione may also be employed in the formulations herein, as a free-radical scavenger.

Neovascular Regulators

Normal angiogenesis regulation appears to be accomplished by a variety of means. Endogenous factors, e.g., body chemistry, genetics, as well as exogenous factors, e.g., types of food consumed, appear to play a role in this important control mechanism. A number of substances have been found to affect angiogenesis. Those substances that inhibit or moderate undesired angiogenesis, particularly angiogenesis linked to disease conditions of the retina (retinopathy), are preferred for use in the compositions of this invention. Preferred compositions of this invention comprise more than one chemical type of angiogenesis regulator or more than one source of an angiogenesis regulator. Different regulators are believed to function in a complimentary manner to achieve a biochemical balance. In addition, components of the compositions, other than specifically listed neovascular agents, may also affect angiogenesis. For example, antioxidants and free-radical scavengers can control free radicals which, by various mechanisms, may destroy angiogenesis regulation. The control of oxidative stress due to antioxidants may have a significant effect on beneficial neovascular control, particularly in the biological states that lead to retinopathy. As discussed above regarding antioxidants, conservative doses of several angiogenic regulators are believed to be more beneficial, i.e., enhanced effectiveness with minimal potential for toxic effect, than larger doses of a single chemical.

Cartilage, an avascular tissue, is a source of angiogenesis inhibitor(s). Shark and bovine cartilage, among others, are sources of angiogenesis inhibitor and may provide collagenase inhibition as well. Chondroitin sulphate, a mucoploysaccharide found in most mammalian cartilaginous tissues and shark cartilage, is believed by many to be the most active angiogenesis regulating component of Shark Cartilage. The restoration of diabetic depleted chondroitin sulphates may also affect collagen stabilization which would help to normalize the collagen matrix of vascular tissue and therefore create a more stable vascular structure. Chondroitin sulphate can be provided in a number of forms with different counterions, e.g., sodium, potassium, etc. Sodium chondroitin sulphate is the form preferred for use in compositions of this invention.

Protamine sulphate is a mixture of the sulphates of basic peptides that can be prepared from the sperm or the mature testes of certain species of fish. It is an arginine rich basic protein which has been shown to be a specific inhibitor of angiogenesis, possibly due to its ability to bind to heparin. Protamine has been used in some insulin preparations to prolong the effects of insulin. Protamine is usually given as the sulphate, but the hydrochloride form may also be used.

Genistein as well as daidzein are plant-derived isoflavonoids, found for example in soybeans, that exhibit an ability to inhibit neovascularization by controlling endothelial cell proliferation in vitro. Soy isolate is a natural source of genistein, daidzein or the glycoside derivatives (e.g., genistin, diadzin and sophoricoside) of these isoflavones. Soy isolate also provides nutritional benefit and may supplement depleted amino acids.

Heparin sulphate levels are increased in diabetics while levels of chondroitin sulphates are decreased. This suggests an imbalance in chondroitin sulphate and in angiogenic regulation. Gymnema Sylvestre which normalizes heparin levels is provided in the compositions of this invention, at least in part, to affect heparin levels which in turn may affect angiogenic regulation due to shark cartilage and protamine sulfate which both bind to heparin. The insulin/glucose stabilization effects of Gymnema sylvestre would reduce the oxidative stress that contributes to the neovascularization factors described above.

Collagen Factors

Restoration of the collagen matrix in vascular and other tissue is an important aspect of the formations of this invention. In this regard, building blocks for collagen synthesis, growth regulators related to collagen synthesis and repair, cofactors for synthesis of collagen, calcium binding and/or regulatory agents and nutrients including various minerals associated with promotion of collagen synthesis are provided in formulas of this invention. Glucosamines stimulate and provide building blocks for collagen synthesis. Chondroitin sulphate is a glucosamine that functions for growth regulation and stimulates collagen synthesis. Glucosamine sulphate is a preferred glucosamine for promoting collagen synthesis and repair.

Manganese is a cofactor which promotes collagen synthesis. Amino acids, particularly branched chain amino acids, provide protein for synthesis of collagen.

Other components that affect collagen synthesis are inhibitors of mammalian collagenases and antioxidants. Inhibition of collagen breakdown by oxidative stress or by enzymatic degradation combined with stimulation and prevention of collagen synthesis is believed to result in improved vascular condition.

Minerals

The compositions of the present invention include various minerals including zinc, chromium, calcium, magnesium, potassium, manganese, and selenium. Optional additives can include other minerals, chromium in non-diabetic formulations, which may have beneficial or nutritional value for a given individual, particularly those minerals that are depleted in a given individual with diabetes. Certain minerals can have additional therapeutic value in the compositions of this invention. For example as discussed above zinc is believed to play a significant role as an antioxidant and many diabetics are found to have a zinc deficiency, especially those with retinopathy.

In general, minerals can be provided in a variety of forms with various counterions. The choice of a given form of mineral will depend generally on the type of dosage form that is employed, whether, for example, an oral or intravenous dosage form is employed. Preferred forms of minerals are generally those that are more absorbable and those that have lower toxicity. In addition, preferred forms will be generally compatible with the other components of a given mixture, will result in minimal irritation or other undesired side effects. Choices of form of a given mineral provided in a given composition of this invention will also depend on the other ingredients in the composition, particularly to avoid excessive levels of a given counter ion.

Zinc can be provided in a variety of forms and with various counter ions, including among others zinc citrate, zinc fumarate, zinc gluconate, zinc alpha-ketoglutarate, zinc lactate, zinc malate, zinc succinate, zinc picolinate or mixtures thereof. The preferred form of zinc in the compositions of this invention is zinc (Krebs) in which the counter ions are a mixture of the anions of the five primary organic acids of the tricarboxylic acid cycle (Krebs Cycle) i.e., a mixture of the zinc salts of citric, fumaric, malic, alpha-ketoglutaric and succinic acids.

Chromium can be provided by a variety of dietary sources including, among others, brewer's yeast, liver, potatoes with skin, beef, fresh vegetables and cheese. Chromium exists in a dinicotino-glutathionine complex in natural foods. Such dietary and natural materials can provide sources of chromium for use in compositions of this invention. As with other minerals there are generally a variety of forms of chromium that are useful in the compositions of this invention including for example, chromium sulphate. Chromium picolinate is particularly preferred for use in this invention because picolinate forms of minerals are generally transported more quickly and efficiently in the body.

Magnesium can be provided in a variety of forms and with various counter ions, including among others magnesium citrate, magnesium fumarate, magnesium gluconate, magnesium alpha-ketoglutarate, magnesium lactate, magnesium malate, magnesium succinate, magnesium picolinate, magnesium sulphate or mixtures thereof. Preferred forms of magnesium in the compositions of this invention are magnesium malate magnesium (Krebs) in which the counter ions are a mixture of the anions of the five primary organic acids of the tricarboxylic acid cycle (Krebs Cycle) i.e., a mixture of the magnesium salts of citric, fumaric, malic, alpha-ketoglutaric and succinic acids.

Calcium can be provided in a variety of forms and with various counter ions, including among others calcium ascorbate, calcium carbonate, calcium citrate, calcium fumarate, calcium gluconate, calcium alpha-ketoglutarate, calcium levulinate, calcium lactate, calcium malate, calcium succinate, calcium picolinate or mixtures thereof. Calcium can also be provided in a variety of natural sources including dolomite, oyster shells, and bone meal. The more preferred form of calcium in the compositions of this invention is calcium (Krebs) in which the counter ions are a mixture of the anions of the five primary organic acids of the tricarboxylic acid cycle (Krebs Cycle) i.e., a mixture of the calcium salts of citric, fumaric, malic, alpha-ketoglutaric and succinic acids. Also preferred for use in compositions of this invention are calcium carbonate, and calcium citrate which are noted for being highly absorbable.

Potassium can be provided in a variety of forms and with various counter ions, including among others potassium citrate, potassium carbonate, potassium fumarate, potassium gluconate, potassium alpha-ketoglutarate, potassium lactate, potassium malate, potassium succinate, potassium picolinate or mixtures thereof. The preferred form of potassium in the compositions of this invention is potassium citrate which has one of the highest levels of elemental potassium.

Manganese, selenium, and strontium can be provided in a variety of forms with various counterions. Selenium is preferably supplied as an organoselenium compound, e.g., selenomethionine. Manganese asparate is a preferred form of manganese for use in the formulas of this invention.

Ranges of zinc (Krebs), calcium (Krebs), magnesium (Krebs), chromium picolinate, potassium citrate and other minerals in an average daily dose of a composition of this invention are provided in Table 3. The ranges given are maximum ranges which may need to be adjusted dependent upon the amount and form of other ingredients included in the composition. These ranges can be readily adjusted by those of ordinary skill in the art of nutrient and therapeutic formulation to other forms of the minerals noted above.

A mineral complex can optionally be combined with the compositions of this invention in addition to or substituted for specific minerals in the various formulas. Preferably, the mineral complex is used to supplement nutritional minerals not already included in specific formulation. A preferred mineral complex includes absorbable salt or chelated forms of:

major mineral components: calcium, magnesium, and potassium also chloride (e.g., as potassium chloride) and sulphate (e.g., as manganese sulphate);

intermediate level components: zinc, manganese, boron and copper;

minor components: chromium, selenium, iodine, molybdenum, vanadium, lithium, rubidium, silicon (as silica), nickel, phosphorus, strontium and cadmium;

trace minerals: preferably from natural sources e.g., marine organic minerals or sea water concentrate.

The minerals may be provided in a variety of salt and complex forms, i.e., as the salts of Krebs cycle acid anions: aspartate, citrate, fumarate, malate and/or succinate salts; as salts of amino acids (e.g. arginates); as picolinate salts; as ascorbate salts, as nicotinate salts. Silicon is preferably provided as the trisillicate anion, e.g. magnesium trisillicate. Selenium is preferably provided as organoselenium compound, e.g. selenomethionine. A variety of natural sources of minerals are known to the art including plant extracts, and can be used to provide minerals in the formula of this invention. A preferred mineral complex is:

Calcium (Krebs, lactate, aspartate, arginate, etc.)

MINERAL COMPLEX

| | | |
|---|---|---|
| Calcium (Krebs) (lactate, aspartate, argininate | 10 mg to | 10,000 mg |
| etc.) | | |
| Magnesium (Krebs), (aspartate, argininate, | 3 mg to | 10,000 mg |
| triscilicate (malate), etc.) | | |
| Potassium (Krebs) (argininate, aspartate) | 2 mg to | 10,000 mg |
| Zinc (Krebs) (picolinate) | 1 mg to | 100 mg |
| Manganese (Krebs) | 10 mcg to | 100 mg |
| Boron (gluconate) | ′0 mcg to | 100 mg |
| Copper (Krebs) | 10 mcg to | 50 mg |
| Chromium (picolinate, nicotinate, etc.) | 2 mcg to | 50 mg |
| Selenium (1-selenomethionine) | 1 mcg to | 50 mg |
| Iodine (marine organic minerals, kelp, etc.) | 1 mcg to | 50 mg |
| Molybdenum (Krebs) | 1 mcg to | 50 mg |
| Vanadium (Krebs) | 1 mcg to | 50 mg |
| Lithium (aspartate, argininate, etc.) | 1 mcg to | 50 mg |
| Rubidium (Krebs) | 1 mcg to | 50 mg |
| Silica (sodium melasilica, magnesium trisilicate) | 10 mcg to | 200 mg |
| Trace minerals (marine organic minerals) | 10 mcg to | 200 mg |
| Cobalt | 10 mcg to | 200 mg |
| Nickel | 1 mcg to | 50 mg |
| Phosphorus (e.g., dicalcium phosphate) | 1 mcg to | 50 mg |
| Chloride (e.g., potassium chloride) | 1 mg to | 1,000 mg |
| Sulphur (manganese sulphate) | 10 mcg to | 100 mg |
| Strontium | 1 mcg to | 800 mg |
| Cadmium | 1 mcg to | 500 mg |

Minerals specifically included in a given formulation of this invention are preferably provided at the level indicated in that formulation. For an individual diagnosed with a particular mineral deficiency (e.g., iron deficiency), dosages of a given mineral may be increased as needed and additional minerals, e.g. iron, may be added to the mineral complex.

Vitamins

Vitamins are included in compositions of this invention to provide supplementation for depletion and dietary deficiencies and in some cases for specific therapeutic benefits. Vitamins may also complement the activity of other components of the composition. Vitamin C, i.e., ascorbic acid, vitamin E, i.e., alpha-tocopherol, and vitamin A provide general nutritional supplementation as well as antioxidant function, as discussed above. Vitamin B6, i.e., pyridoxine, vitamin B12, i.e., cobalamine, and folic acid (folate) provide general nutritional supplementation, and more specific benefits. Folate and vitamins B6 and B12 have antianemia properties. Recent studies suggest that these vitamins may also be helpful in lowering blood levels of homocysteine, an amino acid that has been associated with increased risk of heart disease. Vitamin B2, i.e., riboflavin, provides general nutritional supplementation.

A Vitamin B complex can be employed in addition to or substituted for Vitamin B components of the formulas of this invention. A preferred Vitamin B complex includes:

| | | |
|---|---|---|
| Vitamin B1 (thiamine) | 10 μg-100 mg | (10%)) |
| Vitamin B2 (riboflavin) | 10 μg-50 mg | (5%) |
| Vitamin B3 (nicotinamide or niacinamide, | 1 mg-1,000 mg | (53%) |
| preferably as niacinamide ascorbate) | | |
| Vitamin B5 (pantothenic acid) | 1 mg-200 mg | (26%) |
| Vitamin B6 (pyridoxine HCl) | 10 μg-3 mg | (5%) |
| Vitamin B12 (cyanocobalamin) | 1 μg-200 μg | (0.03%), |

where a preferred range and preferred specific relative amounts of the components are given.

Amino Acids

The formulas of this invention include amino acids that have a particular therapeutic function. Formulas of this invention may also contain additional amino acids for nutrient supplementation or for compensation for an individual's deficiency. Compositions of this invention can include any of the following: alanine, arginine, aspartic acid, cystine, glutamic acid, glycine, histidine, isoleucine, leucine, lysine, methionine, phenylalanine, proline, serine, threonine, tryptophan, tyrosine, valine, carnitine (all in the biologically active L-form) and gamma aminobutryic acid. When present in a given formula, a specifically listed amino acid is preferably provided in the amount needed to provide the desired therapeutic effect. Additional nutritional amino acids are preferably provided in an nutritionally effective amount.

Other Components

Fenugreek (Tigonella foenumgraecum L. Leguminosae) is an annual herb, the seeds of which contain a number of alkaloids, including trigonelline and coumarine, and the steroidal sapogenin, diosgenin. Fenugreek seeds reduce serum cholesterol levels in animals. In particular, the defatted fraction of fenugreek seed which is rich in fiber (about 54%) and contains about 5% of steroidal sapogenin, including diosgenin significantly lowers plasma cholesterol, blood glucose and plasma glucagon levels. Fenugreek is included in certain preferred compositions of this invention for treatment of diabetic complications for its hypoglycemic effect. The preferred form of fenugreek for formulations of this invention is the defatted, fiber-rich fraction.

Source of Omega-3-Fatty Acids

Omega-3 oils are a family of oils having relatively high concentrations of omega-3 polyunsaturated fatty acids, including eicosapentaenoic acid (EPA) and alpha-linolenic acid. These oils exhibit a hypolipidaemic action, especially a reduction in plasma triglycerides linked to a reduction in very-low density lipoproteins (VLDL). They also lower high fibrinogen levels which have been linked to risk of cardiovascular disease. They also exhibit anti-inflammatory and anti-platelet effects. Fish oils and other marine oils typically contain high levels of omega-3-fatty acids. In general, omega-3-fatty acids are believed to reduce blood pressure, and lower cholesterol and triglyceride levels. Omega-3 fatty acids are found in a variety of naturally-occurring sources and may be provided in their acid form or as fatty acid salts or fatty acid esters.

Chronic omega-3-fatty acid deficiency correlates with chronic nephropathic injury. EPA and DHA (docosahexanoic acid) produce an anti-inflammatory effect by reducing prostaglandin production and displacing arachidonic acid. HDL, triglycerides and fibrinogen have also been successfully reduced by omega-3-oils.

Flaxseed (also called Linseed) is a nutrient rich in omega-3-fatty acids. It is a major source of alpha-linolenic acid (an omega-3-fatty acid) and lignin. Ground flaxseed is a preferred source of omega-3-fatty acids over fish oils for use in compositions of this invention. The use of flaxseed oils avoids the potential toxicity that has been associated with long term use of fish oils. Fish and marine oils or individual omega-3-fatty acids, including EPA, and ALA (and their analogous fatty acid esters) can be used in these formulations in place of flaxseed.

EPA ethyl ester has been shown to reduce microalbuminuria in diabetics. Reduction in microalbuminuria may prevent or slow the development of nephropathy.

Essential fatty acids (EFAs) are those fatty acids that cannot be made by the body and must be supplied through the diet. Fresh, poly-unsaturated vegetable oils are a major source for EFAs (linoleic, linolenic and appropriate levels of arachidonic acids). EFAs have a variety of beneficial effects including reduction of blood pressure, lower cholesterol, and lower triglyceride levels. Linolenic acid is the preferred essential fatty acid for formulations of this invention. A natural source of linolenic acid is Evening Primrose Oil which also provides high levels of GLA (about 9%) with minimal toxic properties.

Coenzyme Q₁₀, also designated ubiquinone(50) is one of a group of benzoquinones involved in electron transport. Coenzyme Qₙ, where n=1-12, has a 2,3-dimethoxy-5-methylbenzoquinone nucleus with various terpenoid side chains. Coenzyme Q with 10 isoprene units (Coenzyme Q₁₀) is the most common form in animals. Coenzyme Qₙ, where n=6-10, are naturally occurring. Coenzyme Q₁₀ is a necessary component of the energy-generating process of every cell in the body. Coenzyme Q₁₀ can also function as an antioxidant. Coenzyme Q₁₀, the preferred form of coenzyme Q for human nutrition and therapy, is provided in formulations of the present invention to supplement nutritional deficiencies, particularly in diabetics, which are believed to generally exacerbate disease conditions and cause fatigue. Certain commonly-used oral diabetes drugs, including Tolazamide and Phenformin, may interfere with the enzymes that use Coenzyme Q₁₀, and thus worsen pre-existing deficiencies in diabetics. Adequate tissue reserves of Coenzyme Q₁₀ may also facilitate blood sugar regulation. Coenzyme Q₁₀ is also believed to generally enhance an individual's energy levels. Other forms of coenzyme Q, particularly coenzyme Qₙ, where n is 1-9 and 10-12 and more preferably the naturally-occurring forms where n=6-9, can be employed in place of coenzyme Q₁₀ in the formulas of this invention.

Taurine is found in high concentrations in the brain, retina and kidney cortex. Taurine deficiency has been linked to retinal pathologies. Taurine deficiency has also been found in diabetics. Taurine may have a protective effect on retinal tissue and/or act as an antioxidant. Taurine has been linked to inhibition of platelet aggregation and atherosclerotic lesions and has been found to help control blood pressure. Taurine can be provided from a variety of sources in different forms. Homotaurine, a taurine precursor, is a good bioavailable oral form to provide taurine. Compositions herein can contain taurine or homotaurine.

L-Carnitine is an essential co-factor of fatty acid metabolism. Significantly decreased plasma carnitine levels are common in insulin dependent diabetics including those with nephropathies. This implies that such patients may suffer from inadequate ATP reserves that could cause fatigue and oxidative stress due to reduced lipid metabolism caused by faulty transport of fatty acids across mitochondrial membranes. Carnitine supplementation supports increases in fat utilization and oxygen uptake while decreasing plasma lactate levels and respiratory quotients. Carnitine has been shown to reduce ketones, LDL and triglycerides and increase HDL while acting as a vasodilator. Low carnitine levels may correlate with low plasma albumin and edema. L-Carnitine can be provided as N-acetyl-l-carnitine hydrochloride, the preferred form for this invention. Carnitine can be also be provided as the 1- or d,1-form as hydrochloride or other salts.

Phytosterols, including plant sterols, which comprise beta-sitosterol, campesterol, and/or stigmasterol have been shown to reduce the absorption of the LDL cholesterol component of foods in the gut on a dose dependent basis of approximately one-to-one sterols to cholesterol, while enhancing beneficial HDL to positively effect the LDL-HDL Ratio. An additional benefit of blocking cholesterol absorption is that it frees other ingredients in the formulation of this invention to eliminate existing cholesterol plaque (See Table 4). This reduces the added burden of combating the new plaque development of cholesterol which would not otherwise have been blocked by the plant sterols. Plant sterols have been shown to primarily block harmful LDL cholesterol and admit beneficial HDL cholesterol, the levels of which can actually be elevated. Plant sterols can be provided in the formulas of this invention in soy oil or by addition of individual sterol components. A commercially available mixture of phytosterols, “Cholestatin III” (about 62% beta-sitosterol, about 24% campesterol and about 14% stigmasterol), produced in bacterial fermentation, is preferred for use in the formulas of this invention. Saw palmetto is another useful source of phytosterols.

The inhibition of the absorption of dietary cholesterol can also be enhanced by administration of epigallocatechin gallate found in Green Tea Extract to promote excretion of cholesterol.

Gymnema sylvestre

Gymnemic acid, the active ingredient in Gymnema sylvestre, suppresses sensitivity to sugar and its absorption, thereby reducing blood glucose levels. It also restores the levels of three chondroitin sulfates which may assist in collagen repair and/or aid in angiogenesis regulation. Heparin sulphate levels are increased in diabetics while three chondroitin sulfates are decreased. Gymnema sylvestre which normalizes heparin levels could play a supporting role in the angiogenic regulation of other ingredients in this formulation, namely shark cartilage and protamine sulfate. Both are angiogenic regulators which bind to heparin. The restoration of depleted chondroitin sulfates probably plays a role in collagen stabilization which would help to normalize the collagen matrix and therefore create a more stable structure upon which angiogenesis regulation could more easily exist. The insulin/glucose stabilization effects of Gymnema sylvestre would reduce the oxidative stress that contributes to the neovascularization factors described above.

Allicin is reported to be the active ingredient of garlic and garlic preparations that have been associated with cholesterol and triglyceride reduction. Consumption of garlic has been associated with increased fibrinolysis, reduced platelet aggregation and vasodilation, but clear clinical effect reducing morbidity and mortality in cardiovascular disease has not demonstrated (British Med. J. (1991) 303:379-380; Grunwald, J. (1990) J. British Pharmacol. 28:582-583).

Aloe vera is suggested to be an inhibitor of thromboxane A₂ and useful as an oral and topical agent for wound healing (Davis, R. H. (1989) J Amer. Podiatric Medi. Assoc. 79(11):559-562 and Heggers, J.P. (1993) Phytotherapy Research 7:S48-S52.) Aloe vera is included in oral dosage forms of the formulas of this invention as well as in wound ointment formulation.

Calcitonin (Merck Index, Ninth Edition (1976) 1633 P.208) is a calcium regulating hormone secreted by mammalian thyroid gland that is employed in the treatment of bone disorders including osteoporosis. Amylin (see U.S. Pat. No. 5,405,831) is a peptide found in amyloid deposits of diabetics (Type 2), which may be a peptide hormone having a role in storage and disposal of food as carbohydrate and fat. Amylin increases liver output of glucose, increases lactate production in muscle and decreased insulin action. U.S. Pat. No. 5,405,831 reports that amylin, variants of amylin and amylin agonists are useful, like calcitonin, for the treatment of bone disorders to prevent or inhibit bone resorption because of its role in calcium metabolism.

Centella asiatica is a plant traditionally used in wound healing. An extract, preferably titrated extract (TECA) or total triterpene fraction containing triterpenes, including asiatic acid, can be used in wound healing. Asiatic acid is reported to stimulate collagen synthesis in cell cultures (Maquart, F-X et al. (1990) Connective Tissue Res. 24:107-120 and Tenni, R. et al. (1965) Ital. J. Biochem. 240:3944-3950).

Sulphated saccharides and salts thereof are reported to be useful as an ingredient in topical preparations to the teeth or gingiva for prophylaxis or treatment of diseases of the tooth or tooth-supporting tissue (U.S. Pat. No. 5,240,710). Sulphated saccharides include polysulphated saccharides and persulfated saccharides, for example, sucraflate, which is sucrose octakis (hydrogen sulphate) aluminum complex, or a salt of sucrose octakis (hydrogen sulphate). Polysulfated saccharides have also been suggested to stimulate neovascularization at skin wound sites, but have also been associated with increased inflammation at the wound site (EP 230,023 (1987)).

Vitamin D3 is associated with calcium transport and bone calcium resorption. 1,25-dihydroxy Vitamin D3 is reported to lower blood pressure and increase sensitivity to insulin. Certain analogs and derivatives of 1,25-dihydroxy Vitamin D3 are reported to induce minimal or no hypercalcemia. (Hypercalcemia is a significant contributing factor to the toxicity of Vitamin D's.) Derivatives, such as 22-oxa-Vitamin D3 is thus indicated to have reduced toxicity compared to Vitamin D3. See: Abe, J. et al. (1991)Endrocrinology 129:832-837 and Mark, R. (1992) Pediatric Nephrology 6:345-348. Vitamin D3 is also reported to be important in cell differentiation. The inventor includes Vitamin D3, particularly lower toxicity Vitamin D3 analogs (22-oxa-Vitamin D3) in the formulas of this invention as a calcium regulator that is a factor for promotion of collagen synthesis and more importantly for its additional function in the immune response which is believed will reduce immune attack on endothelial tissue to reduce atherosclerosis and its lesions.

Vitamin K1

Vitamin K is a cofactor involved in blood coagulation. Vitamin K1, or phylloquinone, is a preferred from of Vitamin K for use in the formulas herein. Vitamin K is also reported to increase calcium binding affinity of certain proteins in bone formation. Vitamin K is included in formulas of this invention to supplement any vitamin or cofactor deficiency and for its calcium binding function which indicates usefulness in tissue regeneration. Vitamin K is preferred for addition in formulations for treatment and prevention of dental and gum disorders, particularly gingivitis.

Betain HCl, Pepsin and Sodium Bicarbonate

Inappropriate acidity is believed to be a factor in the pathogenesis of chronic disease. Mitochrondrial antagonism resulting in oxidative stress is a probable mechanism. betain, HCl, pepsin and sodium bicarbonate have all demonstrated the ability to help regulate hyperacidity. In addition, betain HCl and pepsin are among digestive enzymes often deficient in the elderly as well as chronic disease sufferers. Supplementation of these digestive enzymes to those having this deficiency increases the availability of nutrients contained in the food they eat.

The proposed function of components listed in the specific formulas of this invention and stated to be options herein are discussed above, are specified in Tables 1 and 2 or are known to those of ordinary skill in the art.

Table 4 provides compositions of preferred formulations of this invention particularly useful for ameliorating symptoms and conditions that are the complications of diabetes mellitus, including retinopathy and nephropathy. These formulations are further described in Example 1. The specific amounts of given components are listed in the Table as an average daily adult dose. Where appropriate the active amount of a given component, which relates to the amount of active ingredient in the particular component listed, is provided.

Compositions in which the specific daily adult dosage of individual components varies from those listed in Table 4 for the preferred embodiment (or the dosages of active ingredients listed) by less than about 10% are preferred for use in treatment of retinopathy and nephropathy. Compositions in which the specific dosages vary from those listed in Table 4 by less than about 20% are more preferred for use in treatment of retinopathy and nephropathy. The dosages listed in Table 4 were calculated for a preferred dosing schedule of “6 days on, 1 day off” (no nutrient/medication being taken on the seventh day). Dosages can be readily adapted for other dosing schedules by those of ordinary skill in the art. For example, the dosages of Table 4 are reduced by 1/7th for use in a “7 days on” schedule. Preferred dosing schedules of this invention include periodic “days off” the composition to avoid development of the peroxidative state and avoid excessive build-up of antioxidants. Dosing schedules as well as dosage can be readily adjusted for individual needs.

Listed in Table 3 is a broad effective dose range (daily adult dose) for individual active components of the formulas of this invention. The broad dose range given in the table provides guidance regarding approximate minimal effective amounts of given components from any source and guidance for dosage of equivalents. The maximum dosages listed are estimates based generally upon what is known in the art concerning the individual components listed. The maxima listed may merely be based on an estimate of maximum amount that can be practically provided in a daily oral dosage form. Those of ordinary skill in the art will appreciate that the dosages listed in Table 3 are specific for the forms and sources of components listed. Dosages can be readily adapted by those of ordinary skill in the art for use of alternate forms or sources of the components listed or for use of functional equivalents.

Tables 1 and 2 provide a summary of the general biological functions of most components that are believed to be beneficial for the treatment of disorders and conditions associated with macroangiopathy and microangiopathy. This listing provides the inventor's current understanding of the functions provided by components included in the preferred composition and provides guidance for the choice of alternative components with similar functionality. The inventor, however, does not wish to be bound by the specific functional correlations listed in these tables or by proposed functionality of individual activity. The etiology of the diseases and conditions discussed herein is complex and a given component of a formula of this invention may have several different effects. In some cases, the component listed in the table is itself a mixture, for example, pine bark extract is a mixture of naturally occurring compounds. In these cases, different components of the listed mixtures may contribute to different functions listed in Tables 1 and 2.

The compositions of this invention specifically ameliorate diabetic complications including retinopathy and nephropathy. The formulas of this invention are effective in the treatment and prevention of complications associated with both Type I and Type II diabetes. The diagnosis and symptoms of these disorders and complications are understood in the medical arts and a variety of methods are known in the art to evaluate the severity and extent of the conditions. Amelioration of symptoms of retinopathy and nephropathy can be measured by any such methods or procedures known in the art.

The compositions of this invention specifically ameliorate disease conditions of the retina including retinopathy, macular degeneration and cataracts. The diagnosis and symptoms of these disorders and complications are understood in the medical arts and a variety of methods are known in the art to evaluate the severity and extent of the conditions. Amelioration of symptoms of retinal degeneration and related retinal disorders can be measured by any such methods or procedures known in the art.

The compositions of this invention specifically ameliorate neuropathy. The diagnosis and symptoms of this disorder are understood in the medical arts and a variety of methods are known in the art to evaluate the severity and extent of this condition. Amelioration of symptoms of neuropathy can be measured by any such methods or procedures known in the art.

The compositions of this invention specifically ameliorate macrovascular disorders including cardiovascular disease. Cardiovascular disease includes atherosclerosis, the formation of vascular and coronary lesions, and a variety of related conditions. The diagnosis and symptoms of these disorders are understood in the medical arts and a variety of methods are known in the art to evaluate the severity and extent of the conditions. Amelioration of symptoms of cardiovascular disease can be measured by any such methods or procedures known in the art.

The compositions of this invention are useful in the treatment of slow-to-heal or recurrent wounds, specifically those wounds that are associated with diabetes, and specifically those wound in which infection is not the major cause of the failure to heal. The diagnosis and symptoms of this disorder are understood in the medical arts and a variety of methods are known in the art to evaluate the severity and extent of the conditions. Amelioration of recurrent wounds and the increased speed of healing of such wounds can be measured or assessed by any such methods or procedures known in the art.

The compositions of this invention are useful in the treatment and prevention of dental and periodontal disorders, including gingivitis. The diagnosis and symptoms of these disorders are understood in the dental and medical arts and a variety of methods are known in the art to evaluate the severity and extent of the conditions. Amelioration of these disorders can be measured or assessed by any such methods or procedures known in the art.

The following example illustrates this invention and is in no way intended to limit the scope of the invention.

### EXAMPLE 1

A Nutrient and Therapeutic Composition for Improving the Symptoms of Diabetic Retinopathy and Nephropathy

Preferred nutrient and therapeutic composition of this invention are formulas A and B containing the components listed in Table 1 in the dosage amounts listed for “Average Adult Dose Per Day”. The amounts listed are of the active ingredient, unless otherwise noted. The active ingredient may be provided in a variety of forms containing more or less active ingredient than the forms employed specifically in A or B.

The following sources of ingredients listed in Table 1 were employed:

Bilberry extract, as a dry hydroalcohol extract containing anthocyanosides corresponding to 25% (by weight)of anthocyanidines obtained from Indena (Milan, Italy). Grape Seed Extract (leucocyanidins) (90-100% OPCs) was also obtained from Indena (Milan, Italy).

Pine Bark Extract (OPC 90%) was obtained from Euromed (Barcelona, Spain).

Green tea polyphenols (95%, min. 75% catechins, low caffeine) was obtained from TSI, International, Inc. (New York, N.Y.).

N-Acetyl-l-cysteine (99%), L-carnitine base (Product No. 18-1870-00), CoQ10 (ubidecarenone), 1−(+)-ascorbic acid, riboflavin (USP, FCC, Water CAS 7732-18-5 max 1.5%), pyridoxine hydrochloride (USP, FCC), and vitamin B12 (USP) were obtained from Schweizerhall, Inc. (Piscataway, N.J.). Vitamin B12 (cyanocobalamine was diluted in inactive filler to give a 1% by weight mixture). Acetyl-R-carnitine is available from several manufacturers.

Vitamin A acetate (T-500A) was obtained from Hoffmann-La Roche (Belvidere, N.J.).

Taurine (98.5% min.) and folic acid (USP) were obtained from Seltzer Chemicals, Inc. (Carlsbad, Calif.). Homotaurine is available from several manufacturers.

Linoleic Acid (1High Purity, 99% min) was obtained from Spectnun Quality Products (Gardena, Calif.).

Lipoic Acid (99.8%) and protamine sulphate (USP) were obtained from Maypro Industries, Inc. (Harrison N.Y.).

Lutein is provided in a nutrient composition “FloraGlo” Lutien (Trademark, Kemin Industries, Des Moines, Iowa) comprising 5% by weight lutein and 0.22% zeaxanthin. This material is in beadlet form and also comprises vegetable oil, natural vitamin E (as a preservative), rosemary, natural citric acid, gelatin, sucrose and starch. See U.S. Pat. No. 5,382,714.

Chondroitin sulphate as the sodium salt produced by the Strandberg method from beef trachea was obtained from Weinstein Nutritional Products (Irvine, Calif.).

Chromium picolinate “Chromax” was obtained from Nutrition 21 (San Diego, Calif.).

Calcium (Krebs)22%, Zinc (Krebs) 30% and Magnesium (Krebs) were obtained from Monarch Nutritional Laboratories (Ogden, Utah).

Potassium citrate (NF granular) complying with USP, FCC and FAO/WHO Food additive specifications was obtained from Archer Daniels Midland.

Shark cartilage powder (100%, 200 mesh) was obtained from Global Trading (USA) Inc. (Union, N.J.).

Isolated soy protein (“Supro” HD90, Trademark) was obtained from Protein Technologies International (St. Louis, Mo.). Isolate soy protein products from this source are reported to typically contain (in mg/g protein) 0.15 to 0.72 mg daidzein, 0.48 to 1.51 mg genistein, 0.05 to 0.26 glycitein with a total isoflavone content of 0.68 to 2.49 mg (aglucone units adjusted for molecular weight).

Phytosterol complex, “Cholestatin III” can be obtained from several sources.

Vitamin E, d-alpha-tocopheryl acetate (natural source, powder) was obtained from B&D Nutritional Ingredients, Inc. (Carlsbad, Calif.).

Flax seed powder containing about 23 mg of alpha-linolenic acid (omega-3-fatty acid) per 100 grams powder was obtained from Honeyville Grain Inc. (Salt Lake City, Utah).

Fenugreek seed powder was obtained from Botanicals International (Long Beach, Calif.).

Ginkgo biloba L. powder extract about 26% flavonglycosides and Gymnema sylvestre powder were obtained from Motherland International Inc. (Chino, Calif.).

Those of ordinary skill in the art of formulation of nutrients and therapeutic compositions will appreciate that components functionally equivalent to those specifically disclosed herein, as well as alternative forms and sources in addition to those specifically disclosed herein for individual composition ingredients are available. This invention is intended to encompass all such functional equivalents and alternatives that are readily known to the art.

TABLE 1
Summary of Functions of Components of Compositions of
this invention for Microangiopathy and Macroangiopathy

Primary formulas comprise components which:

| | |
|---|---|
| 1. | Function as antioxidant to control oxidative stress; |
| 2. | Function as neovascular regulators controlling angiogenesis to |
| | promote vascular healing and integrity; |
| 3. | Stabilize glucose and amylase factors, for example, to increase |
| | glucose tolerance in diabetes; and |
| 4. | Supplement dietary deficiencies and loss through spillage, particularly |
| | as associated with diabetes. |

Compositions of this invention can further comprise components which:

| 5. | Stabilize insulin supply and decrease sensitivity to glucose; | |
|---|---|---|
| 6. | Stabilize protein factors, control proteinuria, glycosylation and | |
| | albumin; | |
| 7. | Control anti-sclerotic factors, functioning as/to: | |
| | A. | Anti-platelet or anti-thrombic agents |
| | B. | Homocysteine inhibitors |
| | C. | Reduce atherosclerotic lesions |
| | D. | Reduce LDL and VLDL |
| | E. | Improve HDL/LDL ratio |
| | F. | Inhibit lipoprotein (a) production |
| | G. | Inhibit cholesterol absorption in bowel |
| | H. | Enhance cholesterol excretion |
| | I. | Triglycerides inhibitors |
| | J. | Fibrogen inhibitors |
| | K. | Nitric Oxide inhibitors (Optional) |
| | L. | Ketosis regulators |
| 8. | Reduce immune phagocytic response to: | |
| | A. | Leukotrienes, neutrophils, etc. |
| | B. | Immunoglobulin (a) |
| 9. | Reduce and stabilize anti-hypertensives as: | |
| | A. | Angiotensin converting enzyme inhibitors & vasodilators |
| | B. | Prostacyclin inhibitors |
| | C. | Aldose Reductase inhibitors |
| | D. | Blood pressure inhibitor/regulator (systolic only) |
| | E. | Agents to reduce blood pressure during bowel contractions |
| | F. | Anti-edema agent |
| | G. | Histamine suppressors |
| 10. | Enhance cellular or metabolic function, for example for: | |
| | A. | Glutathione restoration |
| | B. | ATP/NAD restoration |
| 11. | Promote vascular healing and integrity by: | |
| | A. | Restoring the collagen matrix |
| | B. | Histamine suppression (Optional) |

| | |
|---|---|
| 12. | Promote better nutrient digestion and absorption |
| 13. | Improve pH factor by controlling digistens and systemic hyperacidity |
| 14. | Participate in collagen synthesis |
| 15. | Calcium regulator |
| 16. | Control myocardial infarction and damage |
| 17. | Increase cardiovascular exercise ability and tolerance |
| 18. | Increase other antioxidants, including Vitamin E, reduced glutathione, |
| | uric acid, superoxide dismutase (SOD), catalyze, or glutathione |
| | peroxidase |
| 19. | Inhibit breakdown of myocardial cell membrane |
| 20. | Provide immune differentiation |
| 21. | Restore Vitamin E levels by intestinal absorption of omega-3-fatty |
| | acids |
| 22. | Improves cell transport and mitochondrial function |
| 23. | Improves sleep for better disease resistance and recovery |
| 24. | Amino acid believed to inhibit or ameliorate diabetes pathogenesis |
| 25. | Amino acid believed to inhibit or ameliorate cardiovascular |
| | pathogenesis |
| 26. | Amino acid believed to contribute to wound healing or prevention |
| 27. | Amino acid believed to inhibit or ameliorate neuropathic pathogenesis |
| 28. | Amino acid believed to inhibit or ameliorate dental and periodontal |
| | pathogenesis |
| 29. | Promoter of DNA polymerase for wound healing |
| 30. | Provides protein sources for wound healing |
| 31. | Contributes to improved bone density |
| 32. | Promotes anti-caries and anti-gingivitis environment |
| 33. | Accelerates wound healing |

TABLE 2
Functions

| Formula Components | Functions Listed in Table 3 |
|---|---|
| Pine Bark Extract | 1, 7D, 8A, 9A, 9F, 9G 14, 32, 33 |
| Bilberry Extract | 1, 9A, 11, 14 |
| Grape Seed Extract | 1, 7D, 8A, 9A, 9F, 9G, 14, 32, 33 |
| Gingko Biloba | 1, 7A, 8A, 9D, 14, 17 |
| Green Tea polyphenols | 1, 3, 7A, 7D, 7E, 7G, 7H, 9A, 9D, 9E, 32 |
| Vitamin C | 1, 4, 6, 7D, 7E, 7F, 9C, 9D, 10A, 14, 18, |
| | 32, 33 |
| Vitamin E | 1, 4, 5, 7D, 9A, 9B, 19, 21, |
| Vitamin A | 1, 4, 7A 7C, 7D, 14 |
| Indole-3-carbinol | 1 |
| Antioxidant carotenoids: | |
| lutein | 1, 4 |
| zeaxanthin | ″ |
| lycopene | 1, 4, 7D, 7E |
| beta carotene | ″ |
| Antioxidant bioflavonoids: | |
| quercitin | 1 |
| rutin | |
| naringin | |
| luteolin | |
| Eugenol (Tulasi Leaf Extract) | 1, 33 |
| L-Taurine (or homotaurine) | 1, 7A, 7C, 9A, 15, 25 |
| L-carnitine (or acetyl-L- | 1, 4, 6, 7D, 7E, 7I, 7L, 9A, 10B, 25 |
| carnitine) | |
| Thioctic acid (α-lipoic acid) | 1, 5 |
| N-acetyl-L-cysteine | 1, 7F |
| Cysteine | 1, 24, 32 |
| Glutathione | 1, 10A |
| CoQ10 | 1, 7A, 22 |
| Creatine phosphate | 1, 19 |
| Chondroitin Sulfate | 2, 11, 14 |
| Glucosamine Sulfate | 2, 6, 11, 14 |
| Cartilage | 2, 11, 14, 30 |
| Soy Isolate | 2, 4 |
| Protamine Sulphate | 2, 11, 14 |
| Vitamin B5 (pantothentic) | 4, 14 |
| Vitamin B1 | 4, 14 |
| Folic Acid | 4, 7B |
| Vitamin B2 | 4, 14 |
| Vitamin B6 | 4, 5, 7B |
| Vitamin B12 | 4, 7B |
| Nicotinamide (Vitamin B3) | 5 |
| B complex^{†} | 4, 7B, 14 |
| Zinc | 1, 3, 4, 5, 15, 29, 31, 32 |
| Magnesium | 3, 4, 5, 7A, 7L, 15, 16, 31 |
| Calcium | 4, 9D, 31 |
| Chromium | 1, 4 |
| Selenium | 1, 4 |
| Potassium | 1, 4, 9D |
| Strontium | 4, 31, 32 |
| Cadmium | 4, 32 |
| Manganese | 4, 14, 31, 32 |
| Silicon | 4, 31, 32 |
| Mineral Complex | 4, etc. |
| Aloe vera | 33 |
| Omega-3-fatty acids | 1, 6, 7J, 8A, 8B |
| Essential fatty acids | 1, 7D |
| Vitamin K1 | 1, 7C, 28, 30, 31, 32 |
| Vitamin D3 | 3, 5, 15, 20 |
| Polysulfated saccharide | 14, 32 |
| Melatonin | 1, 23 |
| Allicin | 7A, 7I, 7J, |
| Phytosterols | 7G |
| Fenugreek Seed (D) | 3, 7D, 7E, 7I |
| Gymnema Sylvestre (D) | 2, 3 |
| L-lysine | 4, 28, 31 |
| L-arginine | 1, 4, 14, 25, 26, 27 |
| Glycme | 6D, 6E, 23, 25, 26 |
| L-alanine | 4, 24 |
| L-methionine | 4, 6D, 6E, 24, 25 |
| L-tryptophan | 4, 23, 24 |
| L-proline | 4, 26 |
| L-tyrosine | 4, 25 |
| Gamma-aminobutryic acid | 23, 25 |
| Branched Chain Amino | 1, 4, 14, 26, 30 |
| Acids* | |
| Betain HCl | 12, 13 |
| Pepsin | 12, 13 |
| Sodium Bicarbonate | 13, 32 |

^{†}B complex = Vit. B1, Vit. B2, Vit. B3, Vit. B5, Vit. B6, and Vit. B12.
*Branched Chain Amino Acids = L-leucine, L-isoleucine, and L-valine.

TABLE 3
Preferred Dosage Ranges for Exemplary Formula Components of this
Invention

| | Average Adult Daily Dose | |
|---|---|---|
| Formula Components | (dose/day) | |
| Pine Bark Extract (<85% OPC) | 3-2,000 | mg |
| Bilberry Extract (25% OPC) | 5-1,500 | mg |
| Grape Seed Extract (95-100% OPC) | 5-2,000 | mg |
| Gingko Biloba (24%) | 5-1,500 | mg |
| Green tea polyphenol | 10-10,000 | mg |
| Vitamin C (ascorbic acid) | 10-5,000 | mg |
| Vitamin E (D-alpha-tocopheryl acetate) | 5-800 | mg |
| Vitamin A | 1,000 IU-25,000 | IU |
| Antioxidant carotenoids: | | |
| lutein | 1-300 | mg |
| zeaxanthin | 1-300 | mg |
| lycopene | 1-300 | mg |
| beta carotene | 10-100,000 | IU |
| Quercitin (and other antioxidant | 1-2,000 | mg |
| bioflavanoids) | | |
| Eugenol (Tulasi leaf extract) | 1-3,000 | mg |
| Taurine (homotaurine) | 5-7,000 | mg |
| Thioctic acid (α-lipoic acid) | 5-1,000 | mg |
| N-acetyl-L-cysteine | 5-3,000 | mg |
| L-cysteine | 1-2,000 | mg |
| Glutathione | 1-1,000 | mg |
| CoQ10 | 4-400 | mg |
| Chondroitin Sulfate | 10-10,000 | mg |
| Glucosamine Sulfate | 10-10,000 | mg |
| Soy Isolate | 50-1,500 | mg |
| Protamine Sulphate | 10-900 | mg |
| Vitamin B5 (pantothentic) | 1-200 | mg |
| Vitamin B1 | 10 μg-100 | mg |
| Folic Acid | 100 μg-1,500 | mg |
| Vitamin B2 (Riboflavin) | 1 μg-50 | mg |
| Vitamin B6 (Pyridoxine HCl) | 1 μg-200 | mg |
| Vitamin B12 (Cyanocobalamin 1%) | 1 μg-100 | mg |
| Nicotinamide (Vitamin B3, nicotinamide | 1-500 | mg |
| ascorbate) | | |
| B complex^{†} | 1-500 | mg |
| Calcium (Krebs) | 10-10,000 | mg |
| Zinc (Krebs) | 10-3,000 | mg |
| Magnesium (Krebs) | 3-10,000 | mg |
| Chromium picolinate | 2 μg-50 | mg |
| Selenium (1-selenomethionine) | 1 μg-50 | mg |
| Potassium citrate | 30-18,000 | mg |
| Strontium | 1 μg-800 | mg |
| Cadmium | 1 μg-500 | mg |
| Manganese (Krebs) | 10 μg-100 | mg |
| Silicon (magnesium trisillicate) | 10 μg-200 | mg |
| Mineral Complex | 1-50,000 | mg |
| Aloe vera (powder) | 10-50,000 | mg |
| Omega-3-fatty acids (flax seed powder) | 10-30,000 | mg |
| Essential fatty acids (linoleic acid) | 10-10,000 | mg |
| Vitamin D3 | 1-10,000 | IU |
| Polysulfated saccharide | 7-10,000 | mg |
| Melatonin | 1-100 | mg |
| L-carnitine (Acetyl-L-carnitine) | 10-3,000 | mg |
| Indole-3-carbinol | 1-1,000 | mg |
| Phytosterols (Cholestatin III) | 10-3,000 | mg |
| Creatine phosphate | 10-20,000 | mg |
| Fenugreek Seed (powder) | 10-30,000 | mg |
| Gymnema Sylvestre | 10-3,000 | mg |
| Vitamin K1 | 15 μg-75 | μg |
| L-lysine | 10-13,000 | mg |
| L-arginine | 10-9,000 | mg |
| L-alanine | 10-12,000 | mg |
| Glycine | 10-9,000 | mg |
| L-methionine | 10-300 | mg |
| L-tryptophan | 10-3,000 | mg |
| L-proline | 10-6,000 | mg |
| L-tyrosine | 10-6,000 | mg |
| Gamma-aminobutryic acid | 10-12,000 | mg |
| Branched Chain Amino Acids* | 10-70,000 | mg |
| Betain HCl | 1-10,000 | mg |
| Pepsin | 1-10,000 | mg |
| Sodium Bicarbonate | 1-10,000 | mg |

^{†}B complex = Vit. B1, Vit. B2, Vit. B3, Vit. B5, Vit. B6, and Vit. B12.
*Branched Chain Amino Acids = L-leucine, L-isoleucine, and L-valine.

TABLE 4
Exemplary Diabetic Compliations Formulation Dosages

| | AVERAGE | AVERAGE |
|---|---|---|
| | ADULT DOSE | ADULT DOSE |
| | PER DAY - | PER DAY - |
| | mg/day | mg/day |
| | FORMULATION | FORMULATION |
| COMPONENT | A | B |
| Bilberry Extract, 25% OPC | 375 | 375 |
| Calcium (Krebs) | 500 | 500 |
| | (110 active) | (110 active) |
| Chondroitin Sulfate | 750 | 750 |
| Chromium Picolinate | 200 μg | 200 μg |
| | (24.60 μg active) | (24.60 μg active) |
| CoQ10 | 20 | 20 |
| Fenugreek Seed Powder | 150 | 150 |
| Flax Seed Powder | 500 | 500 |
| Folic Acid | 800 μg | 450 μg |
| Linoleic Acid | 25 | 25 |
| Ginko Biloba 24% | 25 | 25 |
| Gymnema Sylvestre | 250 | 250 |
| Taurine or Homotaurine | 100 | 100 |
| Grape Seed extract, 95-100% | 100 | 100 |
| OPC | | |
| Acetyl-l-carnitine | 50 | 50 |
| Lutein | 120 | 120 |
| Magnesium (Krebs) | 300 | 300 |
| | (48 active) | (48 active) |
| N-Acetyl-l-cysteine | 200 | 200 |
| Pine Bark Extract (greater than | 20 | 20 |
| 85% OPC) | | |
| Phytosterol Complex (Cholestatin | 200 | 200 |
| III) | | |
| Potassium Citrate | 90 | 90 |
| | (32.4) | (32.4) |
| Protamine Sulfate | 50 | 50 |
| Shark Cartilage 100% | 1,000 | 1,000 |
| Soy Isolate | 1,000 | 1,000 |
| | (920 active) | (920 active) |
| Green Tea Polyphenols | 100 | 100 |
| Lipoic Acid | 20 | 20 |
| Vitamin A | 5,000 iu | 5,000 iu |
| (Acetate Formula A) | | |
| (Palmitate Formula B) | | |
| Vitamin B-2 (Riboflavin) | 3 | 50 |
| Vitamin B-6 (Pyridoxine hydro- | 4.88 active | 213.4 |
| chloride) | (4.0 active) | (175 active) |
| Vitamin B-12 (Cyanocobalamin | 100 μg active | 100 μg active |
| 1%) | | |
| Vitamin C (Ascorbic acid) | 1,000 | 1,000 |
| Vitamin E, d-alpha tocopheryl | 714 | 714 |
| acetate | (500 iu active) | (500 iu active) |
| Zinc (Krebs) | 30 | 30 |
| | (9 active) | (9 active) |

I claim:

1. A composition for amelioration of the symptoms and conditions associated with microangiopathy or macroangiopathy which comprises:
(a) a plant extract having antioxidant effect comprising bioflavonoids in an amount effective for providing said antioxidant effect; and
(b) a neovascular regulator that is an inhibitor of angiogenesis.

2. The composition of claim 1 wherein said neovascular regulator is chondroitin sulfate.

3. The composition of claim 1 which comprises antioxidant bioflavonoid plant extracts from at least two different plant sources.

4. The composition of claim 3 wherein said neovascular regulator is chondroitin sulfate and said composition further comprises a glucosamine.

5. The composition of claim 4 which comprises Pine bark extract in an amount effective for providing an antioxidant effect.

6. The composition of claim 1 for prevention and treatment of diabetic complications of microangiopathy which comprises:
(a) antioxidant components:
Pine bark extract;
Bilberry extract;
Tea polyphenols;
Vitamin C; and
Vitamin E.
in a combined amount effective for providing an antioxidant effect;
(b) Neovascular regulator components chondroitin sulphate and glucosamine sulphate in a combined amount effective for inhibiting angiogenesis and/or stabilization of the collagen matrix; and
(c) absorbable zinc and absorbable chromium in an amount effective for compensation of nutrient deficiency.

7. The composition of claim 1 for prevention and treatment of diabetic complications of microangiopathy which comprises:
(a) antioxidant components:
a plant extract having antioxidant effect;
an antioxidant carotinoid;
an antioxidant flavonoid;
thiotic acid;
Vitamin C;
Vitamin E; and
Vitamin A
in a combined amount effective for providing an antioxidant effect and/or for stimulating collagen synthesis;
(b) neovascular regulators and/or factors for collagen synthesis:
chondroitin sulphate, and
glucosamine sulphate
in a combined amount effective for neovascular regulation and/or stimulating collagen synthesis;
(c) minerals:
absorbable zinc;
absorbable chromium;
absorbable magnesium; and
absorbable calcium
in an amount effective for compensating for nutritional deficiency.

8. The composition of claim 7 further comprising:
Gymnema sylvestre;
Fenugreek seed; and
Ginkgo biloba
each present in an amount effective for providing therapeutic and/or protective function.

9. The composition of claim 8 which comprises the components for formula IJ, each present in an amount effective for providing therapeutic and/or protective function.

10. The composition of claim 1 for wound healing which comprises:
(a) a plant extract having antioxidant effect in an amount effective for providing an antioxidant effect;
(b)) chondroitin sulphate and glucosamine sulphate in a combined amount effective for providing for neovascular regulation and/or for promotion of collagen synthesis;
(c) absorbable magnesium in an amount effective for promotion of collagen synthesis.

11. The composition of claim 10 which comprises:
Pine bark extract;
Grape seed extract;
Tea polyphenols;
chondroitin sulfate;
glucosamine sulfate;
Vitamin C;
absorbable magnesium
each component present in an amount effective for providing therapeutic or protective effect.

12. The composition of claim 11 further comprising aloe vera in an amount effective for producing a benefit for wound healing.

13. The composition of claim 12 further comprising:
Gymnema sylvestre;
Fenugreek seek;
thiotic acid; and
absorbable chromium
each in an amount effective for providing a therapeutic and/or protective effect.

14. A wound healing ointment comprising a composition of claim 10 having the components:
a plant extract having antioxidant effect;
chondroitin sulphate;
Glucosamine sulphate; and
thiotic acid;
each in an amount effective for providing a therapeutic and/or protective effect in a carrier suitable for topical application.

15. The composition of claim 10 which comprises the components of Formula IIG each present in an amount effective for providing a therapeutic and/or protective effect.

16. The composition of claim 1 for treatment and/or prevention of neuropathy which comprises:
(a) a plant extract having antioxidant effect comprising bioflavonoids in an amount effective for providing an antioxidant effect;
(b) a neovascular regulator; and
(c) a source of glucosamine present in a combined amount effective for providing a therapeutic or protective effect.

17. The composition of claim 16 which comprises:
Pine bark extract;
chondroitin sulphate;
glucosamine sulphate;
absorbable magnesium;
absorbable calcium;
thiotic acid;
Ginkgo biloba;
tea polyphenols;
Vitamin C; and
a source of essential fatty acids;
each component present in an amount effective for providing a therapeutic and/or protective effect.

18. The composition of claim 17 further comprising:
Gymnema sylvestre;
Fenugreek seed; and
absorbable chromium.

19. The composition of claim 18 formulated for topical application.

20. A composition according to claim 1 for prevention and/or treatment of cardiovascular disease which comprises:
(a) a plant extract having antioxidant effect comprising bioflavonoids in an amount effective for providing an antioxidant effect;
(b) a neovascular regulator for providing for inhibition of angiogenesis and/or stimulation of collagen synthesis in an amount effective for providing a therapeutic and/or protective effect; and
(c) absorbable zinc present in an amount effective for compensating for nutrient deficiency.

21. The composition of claim 20 which comprises:
Vitamin C;
Vitamin E;
Bilberry Extract;
Pine bark extract;
Tea polyphenols;
soy isolate;
chondroitin sulphate;
Glucosamine sulphate; and
absorbable zinc
each component present in an amount effective for providing a therapeutic and/or protective effect.

22. The composition of claim 21 further comprising:
Gymnema sylvestre;
Fenugreek seed; and
absorbable chromium.

23. A composition for treatment and/or prevention of dental caries and periodontal disease which comprises:
(a) a plant extract having antioxidant effect in an amount effective for providing an antioxidant effect;
(b) absorbable calcium in an amount effective for compensation of nutrient deficiency; and
(c) a Vitamin D3 derivative or analog that induces substantially no hypercalcification in an amount effective for providing a therapeutic and/or protective effect.

24. The composition of claim 23 which comprises:
Pine bark extract;
Tea polyphenols;
absorbable calcium; and
22-oxy-Vitamin D3
each component present in an amount effective for providing a therapeutic and/or protective effect.

25. The composition of claim 24 further comprising:
Gymnema sylvestre;
Fenugreek seed; and
absorbable chromium.

26. The composition of claim 1 further comprising:
ginger;
allicin;
licorice extract;
each present in an amount effective for providing a therapeutic and/or protective effect.

27. A method for treating and/or preventing a symptom condition or disorder associated at least in part with microangiopathy and/or macroangiopathy in an individual having microangiopathy or macroangiopathy which comprises the step of administering to said individual the composition of claim 1.

28. A method for treating and/or preventing symptoms, conditions or disorders associated with diabetic microangiopathy in an individual having diabetic microangiopathy which comprises the step of administering to said individual the composition of claim 6.

29. A method for treatment of slow to heal or recurrent wounds in an individual having such wound which comprises the step of administering to said individual the composition of claim 10.

30. A method for treatment and/or prevention of cardiovascular disease in an individual having such disease or at risk of developing said disease which comprises the step of administering to said individual the composition of claim 20.

31. A method for treatment and/or prevention of neuropathy in an individual having said condition or at risk of developing said condition which comprises administering to said individual the composition of claim 16.

32. A method for treatment and/or prevention of dental caries, periodontal disease and other gum disorders in an individual having such disease or condition which comprises the step of administering to said individual the composition of claim 23.
